SEQUENCE LISTING (2 sequences)

SEQ ID NO: 1 - protein, 512 aa, human MUC13
MKAIIHLTLLALLSVNTATNQGNSADAVTTTETATSGPTVAAADTTETNFPETASTTANTPSFPTATSPAPPIISTHSSSTIPTPAPPIISTHSSSTIPIPTAADSESTTNVNSLATSDIITASSPNDGLITMVPSETQSNNEMSPTTEDNQSSGPPTGTALLETSTLNSTGPSNPCQDDPCADNSLCVKLHNTSFCLCLERYYYNSSTCKKGKVFPGKISVTVSETFDPEEKHSMAYQDLHSEITSLFKDVFGTSVYGQTVILTVSTSLSPRSEMRADDKFVNVTIVTILAETTSDNEKTVTEKINKAIRSSSSNFLNYDLTLRCDYYGCNQTADDCLNGLACDCKSDLQRPNPQSPFCVASSLKCPDACNAQHKQCLIKKSGGAPECACVPGYQEDANGNCQKCAFGYSGLDCKDKFQLILTIVGTIAGIVILSMIIALIVTARSNNKTKHIEEENLIDEDFQNLKLRSTGFTNLGAEGSVFPKVRITASRDSQMQNPYSRHSSMPRPDY

SEQ ID NO: 2 - DNA, 2902 bp, human MUC13
cgggatatcgtcgacccacgcgtccgagcaagaacagctaaaatgaaagccatcattcatcttactcttcttgctctcctttctgtaaacacagccaccaaccaaggcaactcagctgatgctgtaacaaccacagaaactgcgactagtggtcctacagtagctgcagctgataccactgaaactaatttccctgaaactgctagcaccacagcaaatacaccttctttcccaacagctacttcacctgctccccccataattagtacacatagttcctccacaattcctacacctgctccccccataattagtacacatagttcctccacaattcctatacctactgctgcagacagtgagtcaaccacaaatgtaaattcattagctacctctgacataatcaccgcttcatctccaaatgatggattaatcacaatggttccttctgaaacacaaagtaacaatgaaatgtcccccaccacagaagacaatcaatcatcagggcctcccactggcaccgctttattggagaccagcaccctaaacagcacaggtcccagcaatccttgccaagatgatccctgtgcagataattcgttatgtgttaagctgcataatacaagtttttgcctgtgtttagaaaggtattactacaactcttctacatgtaagaaaggaaaggtattccctgggaagatttcagtgacagtatcagaaacatttgacccagaagagaaacattccatggcctatcaagacttgcatagtgaaattactagcttgtttaaagatgtatttggcacatctgtttatggacagactgtaattcttactgtaagcacatctctgtcaccaagatctgaaatgcgtgctgatgacaagtttgttaatgtaacaatagtaacaattttggcagaaaccacaagtgacaatgagaagactgtgactgagaaaattaataaagcaattagaagtagctcaagcaactttctaaactatgatttgacccttcggtgtgattattatggctgtaaccagactgcggatgactgcctcaatggtttagcatgcgattgcaaatctgacctgcaaaggcctaacccacagagccctttctgcgttgcttccagtctcaagtgtcctgatgcctgcaacgcacagcacaagcaatgcttaataaagaagagtggtggggcccctgagtgtgcgtgcgtgcccggctaccaggaagatgctaatgggaactgccaaaagtgtgcatttggctacagtggactcgactgtaaggacaaatttcagctgatcctcactattgtgggcaccatcgctggcattgtcattctcagcatgataattgcattgattgtcacagcaagatcaaataacaaaacgaagcatattgaagaagagaacttgattgacgaagactttcaaaatctaaaactgcggtcgacaggcttcaccaatcttggagcagaagggagcgtctttcctaaggtcaggataacggcctccagagacagccagatgcaaaatccctattcaagacacagcagcatgccccgccctgactattagaatcataagaatgtggaacccgccatggcccccaaccaatgtacaagctattatttagagtgtttagaaagactgatggagaagtgagcaccagtaaagatctggcctccggggtttttcttccatctgacatctgccagcctctctgaatggaagttgtgaatgtttgcaacgaatccagctcacttgctaaataagaatctatgacattaaatgtagtagatgctattagcgcttgtcagagaggtggttttcttcaatcagtacaaagtactgagacaatggttagggttgttttcttaattcttttcctggtagggcaacaagaaccatttccaatctagaggaaagctccccagcattgcttgctcctgggcaaacattgctcttgagttaagtgacctaattcccctgggagacatacgcatcaactgtggaggtccgaggggatgagaagggatacccaccacctttcaagggtcacaagctcactctctgacaagtcagaatagggacactgcttctatccctccaatggagagattctggcaacctttgaacagcccagagcttgcaacctagcctcacccaagaagactggaaagagacatatctctcagctttttcaggaggcgtgcctgggaatccaggaactttttgatgctaattagaaggcctggactaaaaatgtccactatggggtgcactctacagtttttgaaatgctaggaggcagaaggggcagagagtaaaaaacatgacctggtagaaggaagagaggcaaaggaaactgggtggggaggatcaattagagaggaggcacctgggatccaccttcttccttaggtcccctcctccatcagcaaaggagcacttctctaatcatgccctcccgaagactggctgggagaaggtttaaaaacaaaaaatccaggagtaagagccttaggtcagtttgaaattggagacaaactgtctggcaaagggtgcgagagggagcttgtgctcaggagtccagccgcccagcctcggggtgtaggtttctgaggtgtgccattggggcctcagccttctctggtgacagaggctcagctgtggccaccaacacacaaccacacacacacaaccacacacacaaatgggggcaaccacatccagtacaagcttttacaaatgttattagtgtccttttttatttctaatgccttgtcctcttaaaagttattttatttgttattattatttgttcttgactgttaattgtgaatggtaatgcaataaagtgcctttgttagatggtgaaaaaaaaaaaaaaaaaaaa

(19) United States
(12) Patent Application Publication
(10) Pub. No.: US 2004/0092724 A1
(43) Pub. Date: May 13, 2004

(54) Mucin

(76) Inventors: Michael Andrew McGuckin, Coorparoo (AU); Stephanie Jane Williams, Grange (AU)

Correspondence Address: William J McNichol Jr, Reed Smith, 2500 One Liberty Place, 1650 Market Street, Philadelphia, PA, 19103, US

(21) Appl. No.: 10/343,253

(22) PCT Filed: Jul. 27, 2001
(86) PCT No.: PCT/AU01/00930

(30) Foreign Application Priority Data
Jul. 28, 2000 (NZ) 506059

(51) Int. Cl. C07K 14/705; C07H 21/04; C12P 21/02

(52) U.S. Cl. 530/395; 435/69.1; 435/320.1; 435/325; 536/23.5

(57) ABSTRACT

The present invention relates generally to an epithelial mucin. More particularly, the subject invention relates to a new transmembrane mucin and to genetic sequences encoding same, to antibodies directed to the mucin and compositions comprising the mucin, its antibodies or genetic sequences encoding same. The present invention contemplates methods for detecting disease conditions or a propensity for development of disease conditions by screening for aberrations in mucin or its encoding genetic sequence.

### FIELD OF THE INVENTION

The present invention relates generally to an epithelial mucin. More particularly, the subject invention relates to a new transmembrane mucin and to genetic sequences encoding same, to antibodies directed to the mucin and compositions comprising the mucin, its antibodies or genetic sequences encoding same. The present invention contemplates methods for detecting disease conditions or a propensity for development of disease conditions by screening for aberrations in mucin or its encoding genetic sequence.

### BACKGROUND OF THE INVENTION

Bibliographic details of the publications referred to by author in this specification are collected at the end of the description.

Reference to any prior art in this specification is not, and should not be taken as, an acknowledgment or any form of suggestion that this prior art forms part of the common general knowledge in Australia or any other country.

The epithelial mucins are large complex glycoproteins produced by epithelial tissues. Epithelial mucins can now be clearly divided into two distinct sub-families: (a) gel-forming mucins secreted by epithelial goblet cells; and (b) cell surface transmembrane mucins (tm-mucins). The common links between these sub-families include their production by epithelial tissues and the presence of tandem repeat gene sequences that encode heavily O-glycosylated domains.

At least four tin-“epithelial” mucin genes have been identified to date—MUC1, MUC13, MUC4 and MUC12. All four tm-mucins are expressed by various glandular epithelial tissues. MUC13, MUC4 and MUC12 appear to encode very large proteins in comparison to MUC1. Tm-mucins play an important role in barrier function and innate immunity on all ductal and glandular epithelial surfaces. In addition, these molecules probably report on extracellular conditions via their cytoplasmic domains. In addition to transmembrane forms, these genes usually encode secreted forms.

The tm-mucins play important roles in some inflammatory epithelial diseases and in epithelial cancers. This is of clinical significance. For example, MUC1 is highly expressed by most carcinomas and is, therefore, utilized as a serum antigen for monitoring progression of breast and ovarian cancers (Devine et al (1994), McGuckin et al (1995)), is a useful prognostic factor in several different carcinoma types (Ohgami et al (1999), Fujita et al (1999), Hiraga et al (1998), McGuckin et al (1995)), and is currently undergoing intense international scrutiny as a cancer vaccine (Agrawal et al (1998)). Because MUC1 is also shed from the cell surface it finds it way into the blood of patients with these cancers. In contrast, MUC1 is not likely to be shed into the blood from normal epithelial, cells because in these cells it is shed directly into the epithelial lumen.

Mucins are very stable proteins due to their glycosylated domains that protect them from protease digestion. Their stability increases their half-life in the bloodstream as well as being advantageous for processing and storage of samples. Mucins also contain repeating amino acid sequences that are potential epitopes for antibodies, enhancing their detection in antibody-based detection assays. Several different commercial serum assays used to monitor clinical progress in patients with carcinomas utilize measurement of MUC1. For example, CASA (Medical Innovations Ltd, Australia), CA15.3 (Centocor, USA) and Truquant-BR (Biomira, Canada).

Some epithelial tm-mucin genes are also expressed in haematopoietic cells, often in an activation dependent matter. For example, MUC1 is expressed by activated T cells and dendritic cells (McGuckin et al., (2000)], by some B cells and is present at high levels on many myelomas [Treon et al. (1999), Takahashi et al. (1994)], and two to ten percent of bone marrow haemopoietic mononuclear cells [Brugger et al., (1999)]. Tm-mucins may therefore play important roles in a broad range of immunological processes.

The inventors have now identified a novel tm-mucin, which is referred to herein as “MUC13”. MUC13 is the smallest tm-mucin identified to date. The identification of MUC13 permits the development of a range of diagnostic and therapeutic agents.

### SUMMARY OF TECE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier number (SEQ ID NO:). The SEQ ID NOs: correspond numerically to the sequence identifiers <400>1, <400>2, etc. A sequence listing is provided after the claims.

A novel tm-mucin is identified from human tissue and is referred to herein as “MUC13”. The nucleotide and corresponding amino acid sequences of MUC13 are represented by SEQ ID NOS:1 and 2, respectively. The identification of MUC13 permits the development of a range of diagnostic agents including antibodies, probes and primers and therapeutic agents including small molecule modulators and genetic modulators.

Accordingly, one aspect of the present invention provides a protein comprising an amino acid sequence substantially as set forth in SEQ ID NO:1 or a protein which is a functional equivalent or variant thereof and/or a protein which comprises an amino acid sequence having at least about 60% similarity to SEQ ID NO:1 or a homologue, derivative or chemical analogue thereof of said protein.

This protein and its derivatives, homologues and chemical equivalents is referred to herein as “MUC13”.

The present invention further contemplates a composition comprising MUC13 and one or more pharmaceutically acceptable carriers and/or diluents.

Another aspect of the present invention provides a polynucleotide which encodes MUC13.

Accordingly, the present invention provides a polynucleotide which comprises the nucleotide sequence substantially as set forth in SEQ ID NO:2 or a functionally equivalent variant thereof and/or a nucleotide sequence which has at least about 60% similarity to SEQ ID NO:2 and/or a nucleotide sequence capable-of hybridizing to SEQ ID NO:2 or its complementary form under low stringency conditions.

Still a further aspect of the present invention provides antibodies which interact with MUC13.

Preferably, said antibodies do not bind MUC1, MUC13, MUC4 or MUC12.

In another aspect, the present invention provides a probe or primer comprising a nucleic acid molecule sufficiently complementary with the polynucleotide defined in SEQ ID NO:2 or its complement, to bind under low stringency conditions.

In yet a further aspect, the present invention provides a diagnostic kit which includes an antibody, a probe or a primer as defined above.

In still a further aspect, the present invention relates to a method of diagnosing the presence, or of monitoring the progression, of cancer in a patient which employs an antibody, probe or primer as defined above.

In another embodiment of the present invention, there is provided a method of diagnosing the presence, or of monitoring the progression, of an epithelial or haematopoietic malignant or non-malignant disease in a patient which employs an antibody, probe or primer as defined above.

In still a further embodiment, the present invention is directed to a method of defining specific haematopoietic cell populations which employs an antibody, probe or primer as defined above to detect expression of MUC13. The MUC13 expressing cells can then be purified or eliminated from haematopoietic cell populations, including for the purpose of modifying bone marrow cell populations prior to transplantion.

Instill a further embodiment, the-present invention provides a method-of detecting whether a patient has a predisposition to cancer or a related condition which comprises the step of detecting the presence or absence of an alteration in the gene encoding MUC13, wherein the presence of an alteration is indicative of a predisposition to cancer.

In another embodiment, the present invention contemplates a method of detecting whether a patient has a predisposition to an epithelial or haemopoietic malignant or non-malignant disease which comprises the step of detecting the presence or absence of an alteration in the gene encoding MUC13 or of a nucleotide sequence which affects expression of a MUC13 gene, wherein the presence of an alteration is indicative of a predisposition to said epithelial or haemopoietic disease.

Conveniently, the presence or absence of an alteration is determined by analysis of DNA coding for MUC13, such as by comparing the sequence of DNA from a sample from said patient with the DNA sequence coding for wild-type MUC13.

Alternatively, the presence or absence of an alteration is determined by analysis of MRNA transcribed from DNA encoding MUC13, such as by comparing the sequence of mRNA from a sample from said patient with the mRNA sequence transcribed from DNA coding for wild-type MUC13. Probes' and primers may also be used to distinguish between mutated or non-mutated genetic material encoding MUC13.

Yet, a further possibility is that the presence or absence of an alteration is determined by analysis of the amino acid sequence of the expressed MUC13 protein.

The present invention further provides genetically modified animals carrying a mutation in one of both alleles of an equivalent or homologue or relative of the human MUC13 gene.

### BRIEF DESCRIPTION OF THE FIGURES

While the present invention will be understood to be broadly as defined above, it will also be appreciated that it is not limited thereto but that it also includes, embodiments of which the description which follows provides examples. In addition, a better understanding of the subject invention will be gained by reference to the accompanying drawings in which:—

FIG. 1 is a representation of the nucleotide sequence of the MUC13 CDNA (SEQ ID NO:2).

FIG. 2 is a representation of the predicted amino acid sequence of the MUC13 transmembrane glycoprotein (SEQ ID NO:1).

FIG. 3 is a photographic representation of a Northern blot analysis of the MUC13 mRNA.

FIG. 4 is a representation of MUC13 mRNA in 79 normal human tissues.

FIG. 5 is a representation of MUC13 mRNA in normal tissues and cancers of the colon.

FIG. 6 is a representation of MUC13 mRNA in normal tissues and cancers of the rectum.

FIG. 7 is a representation of. MUC13 mRNA in normal tissues and cancers of the stomach.

FIG. 8 is a representation of MUC13 mRNA in normal tissues and cancers of the oesophagus.

FIG. 9 is a representation of MUC13 mRNA in normal tissues and cancers of the ovary.

FIG. 10 is a representation of MUC13 MRNA in normal tissues and cancers of the bladder.

FIG. 11 is a representation of MUC13 mRNA in breast cancers.

FIG. 12 is a representation of MUC13 to human chromosome 3q13.3 using fluorescence in situ hybridization with a cDNA probe corresponding to bases 688-1770 in FIG. 1.

FIG. 13 is a representation of MUC13 mRNA in normal tissues and cancers of the kidney.

### DETAILED DESCRIPTION OF THE PREFERRED EMBODIMENTS

The present invention is generally directed to a human tm-mucin and to animal and in particular mammalian homologues thereof. This mucin is referred to herein as “MUC13”. Reference herein to MUC13 includes all equivalents, homologues, derivatives and chemical analysis thereof. The gene encoding MUC13 is referred to herein as “MUC13”. Reference herein to MUC13 includes all equivalents, homologues, derivatives and chemical analogues thereof A “derivative” includes single or multiple amino acid or nucleotide substitutions, deletions and/or additions or inversions as discussed further in the specification.

The present invention contemplates, therefore, a MUC13 protein, generally in isolated form, comprising an amino acid sequence substantially as set forth in SEQ ID NO:1 or an amino acid sequence having at least about 60% similarity thereto after optimal alignment. Such a protein contemplated by the present invention includes derivatives and polymorphisms of MUC13 whether naturally-occurring or artificially generated. Natural mutants or those induced by environmental or industrial carcinogens are proposed to contribute to disease conditions, such as cancer or epithelial or haematopoietic malignant or non-malignant disease conditions.

In accordance with the present invention, MUC13 has been established as described hereinafter.

At the N-terminus is a signal peptide for the secretory pathway with cleavage predicted between residues 19 and 20. The signal peptide is followed by a serine and threonine rich domain likely to involve extensive O-glycosylation (amino acid residues 20-170) consisting of ten degenerate tandem repeats. Following this mucin domain are two distinct cysteine-rich domains containing EGF-like motifs. Separating the two cysteine-rich domains are 115 amino acids comprising a SEA module (amino acid residues 212-328). The first cysteine-rich domain contains one EGF-like sequence EGF1 (amino acid residues 177-210), and the second larger cysteine-rich domain contains two EGF-like sequences, EGF2 (amino acid residues 326-360) and EGF3 (amino acid residues 367-403). EGF3 contains a type II EGF signature (amino acid residues 389-403) and is, followed closely by a 23 amino acid transmembrane domain and a 69 amino acid cytoplasmic tail. In addition to extensive potential O-glycosylation sites in the mucin domain, there are six extracellular and one intracellular consensus motifs for N-glycosylation. The cytoplasmic tail contains a protein kinase C consensus phosphorylation motif (amino acid residues 444-447), and eight serine residues and two tyrosine residues that may undergo phosphorylation and regulate MUC13 signalling.

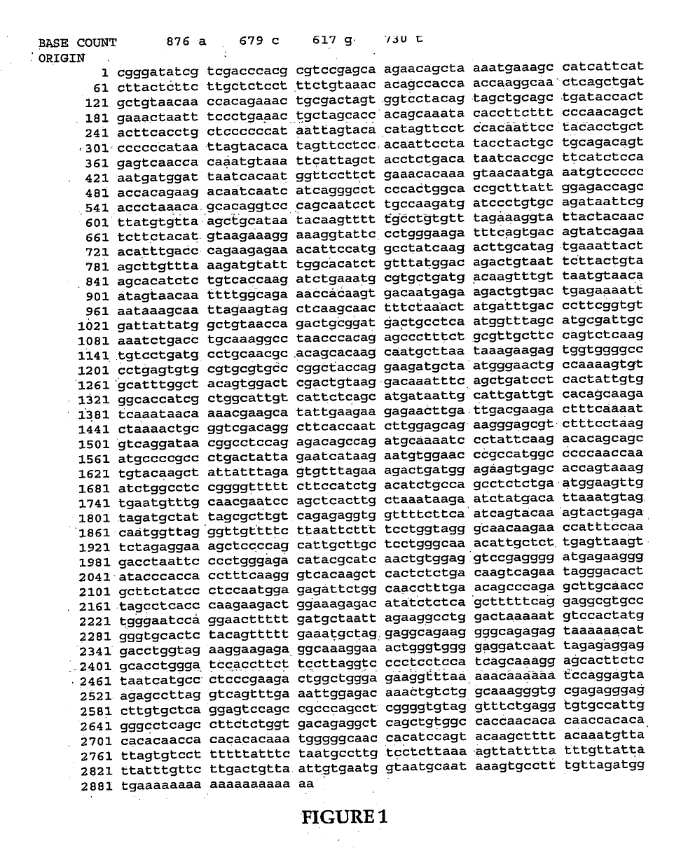

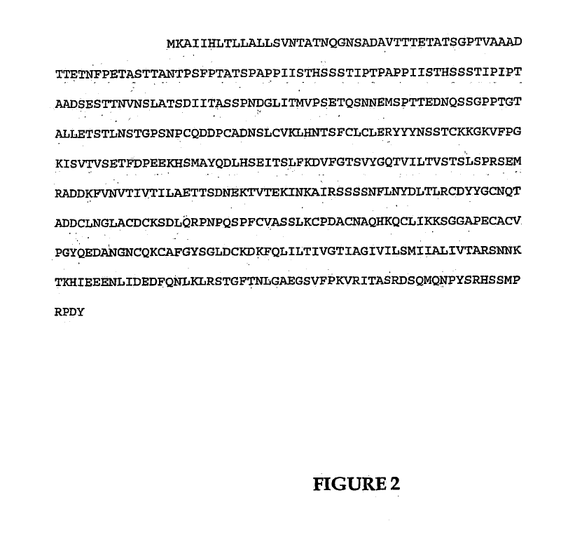

The DNA sequence and predicted amino acid sequence of MUC13 is shown in FIGS. 1 and 2, respectively and are represented in SEQ ID NOS:2 and 1, respectively.

The present invention further contemplates a nucleic acid molecule, generally in isolated form or in a vector comprising a sequence of nucleotides substantially as set forth in SEQ ID NOS:1 or 2 or a nucleotide sequence having at least 0.60% similarity thereto after optimal alignment or a nucleotide sequence capable of hybridizing to SEQ ID NO:2 or its complementary form under low stringency conditions. This sequence corresponds to MUC13 or its derivatives or homologues. Preferred sequence similarities include from about 65% or about 70% or about 80% or about 90% or about 95% or above such as 96%, 97%, 98% or 99%.

The term “similarity” as used herein includes exact identity between compared sequences at the nucleotide or amino acid level. Where there is non-identity at the nucleotide level, “similarity” includes differences between sequences which result in different amino acids that are nevertheless related to each other at the structural, functional, biochemical and/or conformational levels. Where there is non-identity at the amino acid level, “similarity” includes amino acids that are nevertheless related to each other at the structural, functional, biochemical and/or conformational levels. In a particularly preferred embodiment, nucleotide and sequence comparisons are made at the level of identity rather than similarity.

Terms used to describe sequence relationships between two or more polynucleotides or polypeptides include “reference sequence”, “comparison window”, “sequence sirnilarity”, “sequence identity”, “percentage of sequence similarity”, “percentage of sequence identity”, “substantially similar” and “substantial identity”. A “reference sequence” is at least 12 but frequently 15 to 18 and often at least 25 or above, such as 30 monomer units, inclusive of nucleotides and amino acid residues, in length. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e. gaps) of about 20% or less as compared to the sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as, for example, disclosed by Altschul et al. (1997). A detailed discussion of sequence analysis can be found in Unit 19.3 of Ausubel et al. (1998).

The terms “sequence similarity” and “sequence identity” as used herein refers to the extent that sequences are identical or functionally or structurally similar on a nucleotide-by-nucleotide basis or an amino acid-by-amino acid basis over a window of comparison. Thus, a “percentage of sequence identity”, for example, is calculated by comparing two optimally aligned sequences over the window of comparison, determining the number of positions at which the identical nucleic acid base (e.g. A, T, Cy G, I) or the identical amino acid residue (e.g. Ala, Pro, Ser, Thr, Gly, Val, Leu, Ile, Phe, Tyr, Trp, Lys, Arg, His, Asp, Glu, Asn, Gin, Cys and Met) occurs in both sequences to yield the number of matched positions, dividing the number of matched positions by the total number of positions in the window of comparison (i.e., the window size), and multiplying the result by 100 to yield the percentage of sequence-identity. For the purposes of the present invention; “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software. Similar comments apply in relation to sequence similarity.

Reference herein to a low stringency includes and encompasses from at least about 0 to at least about 15% v/v formamide and from at least about 1 M to at least about 2 M salt for hybridization, and at least about 1 M to at least about 2 M salt for washing conditions. Generally, low stringency is at from about 25-30° C. to about 42° C. The temperature may be altered and higher temperatures used to replace formamide and/or to give alternative stringency conditions. Alternative stringency conditions may be applied where necessary, such as medium stringency, which includes and encompasses from at least about 16% v/v to at least about 30% v/v formamide and from at least about 0.5 M to at least about 0.9 M salt for hybridization, and at least about 0.5 M to at least about 0.9 M salt for washing conditions, or high stringency, which includes and encompasses from at least about 31% v/v to at least about 50% v/v formamide and from at least about 0.01 M to at least about 0.15 M salt for hybridization, and at least about 0.01 M to at least about 0.15 M salt for washing conditions. In general, washing is carried out Tₘ=69.3+0.41 (G+C)% (Marmur and Doty, 1962). However, the Tₘ of a duplex DNA decreases by 1° C. with every increase of 1% in the number of mismatch base pairs (Bonner and Laskey, 1974). Formamide is optional in these hybridization conditions. Accordingly, particularly preferred levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

It will be understood, therefore, that the present invention is not limited in scope to molecules which have the specific sequences of FIGS. 1 and 2, but that it also includes functional equivalents of MUC13 or MUC13 as well as derivatives thereof, whether functional or not. Non-functional derivatives may also be useful for generating antibodies as antagonists or in diagnostic kits. Functional equivalents include variant proteins. A protein is considered a functional equivalent of another protein for a specific function if the equivalent protein is immunologically cross-reactive with, and has the same function as, the original protein. The equivalent may, for example, be a fragment of the protein, a fusion of the protein or fragment with other amino acids or a substitution, addition or deletion mutant of the protein.

For example, it is possible to substitute amino acids in a sequence with equivalent amino acids using conventional techniques. Groups of amino acids known normally to be equivalent are:—

| | |
|---|---|
| (a) | Ala (A) Ser (S) Thr (T) Pro (P) Gly (G); |
| (b) | Asn (N) Asp (D) Glu (E) Gln (Q); |
| (c) | His (H) Arg (R) Lys (K); |
| (d) | Met (M) Leu (L) Ile (I) Val (V); and |
| (e) | Phe (F) Tyr (Y) Trp (W). |

Substitutions, additions and/or deletions in MUC13 may be made as long as the resulting equivalent protein is immunologically cross-reactive with, and has the same function as, the native MUC13.

The equivalent MUC13 will normally have substantially the same amino acid sequence as, the native MUC13. An amino acid sequence that is substantially the same as another sequence, but that differs from the other sequence by means of one or more substitutions, additions and/or deletions is specifically considered to be an equivalent sequence. Preferably, less than 25%, more preferably less than 10%, and most preferably less than 5% of the number of amino acid residues in the amino acid sequence of the native MUC13 are substituted for, added to, or deleted from.

Functionally equivalent polynucleotides which encode a protein having MUC13 functionality are also contemplated.

Such equivalent polynucleotides include nucleic acid sequences that encode proteins equivalent to MUC13 as defined above. Equivalent nucleic acid molecules also include nucleic acid sequences that, due to the degeneracy of the nucleic acid code, differ from native nucleic acid sequences in ways that do not affect the corresponding amino acid sequences.

Functionally equivalent proteins and polynucleotides can also be identified with the assistance of computer algorithms that are publicly available. These include BLASTN and BLASTP, which are accessible on the NCBI anonymous FTP server (ftp://ncbi.nlm.nih.gov) under /blast/executables/. The use of the BLAST family of algorithms, including BLASTN and BLASTP, is described at NCBI's website at URL httpl/www.ncbi.nlm.nih.gQvIBLAST/newblast.html and in the publication of Altschul et al. (1997).

MUC13 and its functional equivalents may be prepared by methods known in the art. Such methods include protein synthesis from individual amino acids as described by Stuart and Young in “Solid Phase Peptide Synthesis”, 2^{nd} Edition, Pearce Chemical Company (1984). It is, however, preferred that transmembrane MUC13 and/or its functional equivalents be prepared by recombinant methods. Such methods involve insertion of polynucleotides encoding the desired protein into appropriate expression vectors using art standard techniques such as are described in Sambrook et al., “Molecular Cloning”, 2^{nd} Edition, Cold Spring Harbour Laboratory, Cold Spring Harbour; New York (1987).

The present invention further contemplates chemical analogues of MUC13. Analogues of the MUC13 contemplated herein include, but are not limited to, modifications of side chains, incorporation of unnatural amino acids and/or their derivatives during peptide, polypeptide or protein synthesis and the use of crosslinkers and other methods which impose conformational constraints on the proteinaceous molecule or their analogues.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid CPNS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitization, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, -t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acid, contemplated herein is shown in Table 3.

These types of modifications may be important to stabilize the complex if administered to an individual or for use as a diagnostic reagent.

Crosslinkers can be used, for example, to stabilize 3D conformations, using homo-bifunctional crosslinkers such as the bifunctional imido esters having (CH₂), spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N_{α}-methylamino acids, introduction of double bonds between C_{α} and C_{β} atoms of amino acids and the formation of cyclic peptides or analogues by introducing covalent bonds such as forming an amide bond between the N and C termini, between two side chains or between a side chain and the N or C terminus.

TABLE 1

| Non-conventional | | Non-conventional | |
|---|---|---|---|
| amino acid | Code | amino acid | Code |
| α-aminobutyric acid | Abu | L-N-methylalanine | Nmala |
| α-amino-α-methyl- | Mgabu | L-N-methylarginine | Nmarg |
| butyrate | | | |
| aminocyclopropane- | Cpro | L-N-methylasparagine | Nmasn |
| carboxylate | | L-N-methylaspartic acid | Nmasp |
| aminoisobutyric | Aib | L-N-methylcysteine | Nmcys |
| acid | | | |
| aminonorbornyl- | Norb | L-N-methylglutamine | Nmgln |
| carboxylate | | L-N-methylglutamic acid | Nmglu |
| cyclohexylalanine | Chexa | L-Nmethylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methyl- | Dmasn | α-methyl-α-napthylalanine | Manap |
| asparagine | | | |
| D-α-methylaspartate | Dmasp | α-methylpenicillamine | Mpen |
| D-α-methylcysteine | Dmcys | N-(4-aminobutyl)glycine | Nglu |
| D-α-methyl- | Dmgln | N-(2-aminoethyl)glycine | Naeg |
| glutamine | | | |
| D-α-methylhistidine | Dmhis | N-(3-aminopropyl)glycine | Norn |
| D-α-methyliso- | Dmile | N-amino-α-methylbutyrate | Nmaabu |
| leucine | | | |
| D-α-methylleucine | Dmleu | α-napthylalanine | Anap |
| D-α-methyllysine | Dmlys | N-benzylglycine | Nphe |
| D-α-methyl- | Dmmet | N-(2-carbamylethyl)glycine | Ngln |
| methionine | | | |
| D-α-methyl- | Dmorn | N-(carbamylmethyl)glycine | Nasn |
| ornithine | | | |
| D-α-methylphenyl- | Dmphe | N-(2-carboxyethyl)glycine | Nglu |
| alanine | | | |
| D-α-methylproline | Dmpro | N-(carboxymethyl)glycine | Nasp |
| D-α-methylserine | Dmser | N-cyclobutylglycine | Ncbut |
| D-α-methyl- | Dmthr | N-cycloheptylglycine | Nchep |
| threonine | | | |
| D-α-methyl- | Dmtrp | N-cyclohexylglycine | Nchex |
| tryptophan | | | |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methyl- | Dnmasn | N-cycloundecylglycine | Ncund |
| asparagine | | | |
| D-N-methyl- | Dnmasp | N-(2,2-diphenylethyl)gly- | Nbhm |
| aspartate | | cine | |
| D-N-methylcysteine | Dnmcys | N-(3,3-diphenylpropyl)gly- | Nbhe |
| | | cine | |
| D-N-methyl- | Dnmgln | N-(3-guanidinopropyl)gly- | Narg |
| glutamine | | cine | |
| D-N-methyl- | Dnmglu | N-(1-hydroxyethyl)glycine | Nthr |
| glutamate | | | |
| D-N-methylhistidine | Dnmhis | N-(hydroxyethyl))glycine | Nser |
| D-N-methyliso- | Dnmile | N-(imidazolylethyl))glycine | Nhis |
| leucine | | | |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclo- | Nmchexa | D-N-methylmethionine | Dnmmet |
| hexylalanine | | | |
| D-N-methyl- | Dnmorn | N-methylcyclopentylalanine | Nmcpen |
| ornithine | | | |
| N-methylglycine | Nala | D-N-methylphenylalanine | Dnmphe |
| N-methylaminoiso- | Nmaib | D-N-methylproline | Dnmpro |
| butyrate | | | |
| N-(1-methyl- | Nile | D-N-methylserine | Dnmser |
| propyl)glycine | | | |
| N-(2-methyl- | Nleu | D-N-methylthreonine | Dnmthr |
| propyl)glycine | | | |
| D-N-methyl- | Dnmtrp | N-(1-methylethyl)glycine | Nval |
| tryptophan | | | |
| D-N-methyl- | Dnmtyr | N-methyla-napthylalanine | Nmanap |
| tyrosine | | | |
| D-N-methylvaline | Dnmval | N-methylpenicillamine | Nmpen |
| γ-aminobutyric | Gabu | N-(p-hydroxyphenyl)gly- | Nhtyr |
| acid | | cine | |
| L-t-butylglycine | Tbug | N-(thiomethyl)glycine | Ncys |
| L-ethylglycine | Etg | penicillamine | Pen |
| L-homophenyl- | Hphe | L-α-methylalanine | Mala |
| alanine | | | |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methyl- | Mgln | L-α-methylglutamate | Mglu |
| glutamine | | | |
| L-α-methyl- | Mhis | L-α-methylhomophenyl- | Mhphe |
| histidine | | alanine | |
| L-α-methyliso- | Mile | N-(2-methylthioethyl)gly- | Nmet |
| leucine | | cine | |
| L-α-methyl- | Mleu | L-α-methyllysine | Mlys |
| leucine | | | |
| L-α-methyl- | Mmet | L-α-methylnorleucine | Mnle |
| methionine | | | |
| L-α-methyl- | Mnva | L-α-methylornithine | Morn |
| norvaline | | | |
| L-α-methylphenyl- | Mphe | L-α-methylproline | Mpro |
| alanine | | | |
| L-α-methylserine | Mser | L-α-methylthreonine | Mthr |
| L-α-methyl- | Mtrp | L-α-methyltyrosine | Mtyr |
| tryptophan | | | |
| L-α-methylvaline | Mval | L-N-methylhomophenyl- | Nmhphe |
| | | alanine | |
| N-(N-(2,2-diphenyl- | Nnbhm | N-(N-(3,3-diphenyl- | Nnbhe |
| ethyl)carbamyl- | | propyl)carbamyl- | |
| methyl)glycine | methyl)glycine | | |
| 1-carboxy-1-(2,2- | Nmbc | | |
| diphenylethyl- | | | |
| amino)cyclopropane | | | |

Other derivatives contemplated by the present invention include a range of glycosylation variants from a completely unglycosylated molecule to a modified glycosylated molecule. Altered glycosylation patterns may result from expression of recombinant molecules in different host cells.

The present invention further contemplates compositions comprising MUC13 or modulatory agents thereof. The invention provides, therefore, the use of a MUC13 modulatory agent of MUC13 in compositions for treatment or prophylaxis of a cancer or tumour or other MUC13-related condition. The invention, therefore, also extends to a method for treating or preventing a disease condition comprising administering to a patient in need of such treatment an effective amount of a modulatory agent. A modulatory agent may be an agonist or antagoist, depending on the condition being treated.

A pharmaceutical composition according to the invention is adminstered to a patient, preferably prior to such symptomatic state associated with, for example; the cancer or tumour. The therapeutic agent present in the composition is provided for a time and in a quantity sufficient to treat that patient. Suitably, the pharmaceutical composition comprises a pharmaceutically acceptable carrier.

Depending on the specific conditions being treated, therapeutic agents may be formulated and administered systemically or locally. Techniques for formulation and administration may be found in “Remington's Pharmaceutical Sciences,” Mack Publishing Co., Easton, Pa., latest edition. Suitable routes may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. For injection, the therapeutic agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art. Intra-muscular and subcutaneous injection is appropriate, for example, for administration of immunogenic compositions, vaccines and DNA vaccines.

The agents can be formulated readily using pharmaceutically acceptable carriers well known in the art into dosages suitable for oral administration. Such carriers enable the compounds of the invention to be formulated in dosage forms such as tablets, pills, capsules, liquids, gels, syrups, slurries, suspensions and the like, for oral ingestion by a patient to be treated. These carriers may be selected from sugars, starches, cellulose and its derivatives, malt, gelatine, talc, calcium sulphate, vegetable oils, synthetic oils, polyols, alginic acid, phosphate buffered solutions, emulsifiers, isotonic saline, and pyrogen-free water.

Pharmaceutical compositions suitable for use in the present invention include compositions wherein the active ingredients are contained in an effective amount to achieve its intended purpose. The dose of agent administered to a patient should be sufficient to effect a beneficial response in the patient over time such as a reduction in the symptoms associated with the cancer or tumour. The quantity of the agent(s) to be administered may depend on the subject to be treated inclusive of the age, sex, weight and general health condition thereof. In this regard, precise amounts of the agent(s) for administration will depend on the judgement of the practitioner. In determining the effective amount of the agent to be administered in the treatment or prophylaxis of the condition, the physician may evaluate tissue levels of a polypeptide, fragment, variant or derivative of the invention, and progression of the disorder. In any event, those of skill in the art may readily determine suitable dosages of the therapeutic agents of the invention.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilisers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl-pyrrolidone (PVP). If desired, disintegrating agents may be added, such as the cross-linked polyvinyl pyrrolidone, agar, or alginic acid or a salt thereof such as sodium alginate. Such compositions may be prepared by any of the methods of pharmacy but all methods include the step of bringing into association one or more therapeutic agents as described above with the carrier which constitutes one or more necessary ingredients. In general, the pharmaceutical compositions of the present invention may be manufactured in a manner that is itself known, e.g. by means of conventional mixing, dissolving, granulating, dragee-making, levigating, emulsifying, encapsulating, entrapping or lyophilizing processes.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical which can be used orally include push-fit capsules made of gelatin, as well as soft, sealed capsules made of gelatin and a plasticiser, such as glycerol or sorbitol. The push-fit capsules can contain the active ingredients in admixture with filler such as lactose, binders such as starches, and/or lubricants such as talc or magnesium stearate and, optionally, stabilisers. In soft capsules, the active compounds may be dissolved or suspended in suitable liquids, such as fatty oils, liquid paraffin, or liquid polyethylene glycols. In addition, stabilizers may be added.

Dosage forms of the therapeutic agents of the invention may also include injecting or implanting controlled releasing devices designed specifically for this purpose or other forms of implants modified to act additionally in this fashion. Controlled release of an agent of the invention may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, controlled release may be effected by using other polymer matrices, liposomes and/or microspheres.

Therapeutic agents of the invention may be provided as salts with pharmaceutically compatible counterions. Pharmaceutically compatible salts may be formed with many acids, including but not limited to hydrochloric, sulphuric, acetic, lactic, tartaric, malic, succinic, etc. Salts tend to be more soluble in aqueous or other protonic solvents that are the corresponding free base forms.

For any compound used in the method of the invention, the therapeutically effective dose can be estimated initially from cell culture assays. For example, a dose can be formulated in animal models to achieve a circulating concentration range that includes the IC50 as determined in cell culture (e.g. the concentration of a test agent, which achieves a half-maximal inhibition or enhancement of MUC13 activity). Such information can be used to more accurately determine useful doses in humans.

Toxicity and therapeutic efficacy of such therapeutic agents can be determined by standard pharmaceutical procedures in cell cultures or experimental animals, e.g. for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration utilized. The exact formulation, route of administration and dosage can be chosen by the individual physician in view of the patient's condition. (See for example Fingl et al., 1975).

Dosage amount and interval may be adjusted individually to provide plasma levels of the active agent which are sufficient to maintain MUC13-modulatory-inhibitory effects. Usual patient dosages for systemic administration range from 1-2000 mg/day, commonly from 1-250 mg/day, and typically from 10-150 mg/day. Stated in terms of patient body weight, usual dosages range from 0.02-25 mg/kg/day, commonly from 0.02-3 mg/kg/day, typically from 0.2-1.5 mg/kg/day. Stated in terms of patient body surface areas, usual dosages range 201 from 0.5-1200 mg/m/day, commonly from 0.5-150 mg/day, typically from 5-100 mg/m²/day.

Alternately, one may administer the compound in a local rather than systemic manner, for example, via injection of the compound directly into a tissue, which is preferably a kidney tissue, a stomach tissue or a rectal tissue, often in a depot or sustained release formulation. Furthermore, one may administer the drug in a targeted drug delivery system, for example, in a liposome coated with tissue-specific antibody. The liposomes will be targeted to and taken up selectively by the tissue. In cases of local administration or selective uptake, the effective local concentration of the agent may not be related to plasma concentration.

Genetic vaccines may also be administered. Such composition may comprise nucleic acid molecules encoding MUC13 or genetic molecules which modulate expression of MUC13 such as antisense molecules, co-suppression molecules or ribozymes.

Antibodies to MUC13 are also provided by the present invention. Such antibodies may be monoclonal or polyclonal but monoclonal antibodies are preferred. These can be raised to separate regions of MUC13. Specifically, antibodies can be raised against the cytoplasmic domain, transmembrane domain, the EGF-like extracellular domains, the domain bridging the EGF-like domains, and the N-terminal mucin domain.

Human or non-human monoclonal antibodies are encompassed by the present invention. Where the antibodies are required for administration to a human, a de-immunized or humanized form of a non-human antibody is contemplated by the present invention.

Monoclonal antibodies with affinities of 10⁻⁸ M⁻¹ or preferably 10⁻⁹ to 10¹⁰ M⁻¹ or stronger are typically made by standard procedures as described, e.g. in Harlow & Lane (1988) or Goding (1986). Briefly, appropriate animals will be selected and the desired immunization protocol followed. After the appropriate period, of time, the spleens of such, animals are excised and individual spleen cells fused, typically, to immortalised myeloma cells under appropriate selection conditions. Thereafter, the cells are clonally separated and the supernatants of each clone tested for their production of an appropriate antibody specific for the desired region of the antigen.

Other suitable techniques for preparing antibodies well known in the art involve in vitro exposure of lymphocytes to the antigenic polypeptides, or alternatively, to selection of libraries of antibodies in phage or similar vectors.

Also, recombinant immunoglobulins may be produced using procedures known in the art (see, for example, U.S. Pat. No. 4,816,567 and Hodgson J. (1991)).

The antibodies may be used with or without modification. Frequently, antibodies will be labeled by joining, either covalently or non-covalently, a substance which provides for a detectable signal. A wide variety of labels and conjugation techniques are known and are reported extensively in the literature. Suitable labels include radionuclides, enzymes, substrates, cofactors, inhibitors, fluorescent agents, chemiluminescent agents, magnetic particles and the like. Patents teaching the use of such labels include U.S. Pat. Nos. 3,817,837, 3,850,752, 3,939,350, 3,996,345, 4,277,437, 4,275,149 and 4,366,241.

The immunological assay in which the antibodies are employed can involve any convenient format known in the art. Such formats include Western blots, immunohistochemical assays and ELISA assays. Formats equivalent to those adopted for CASA, CA15.3 and Truquant-BR can also be employed.

Illustrative assay strategies which can be used to detect a target protein of the invention include, but are not limited to, immunoassays involving the binding of an antigen-binding molecule to the target protein (e.g. MUC13 protein) in the sample and the detection of a complex comprising the antigen-binding molecule and the target polypeptide. Preferred immunoassays are those that can measure the level and/or functional activity of a target molecule of the invention. Typically, an antigen-binding molecule that is immunointeractive with a target polypeptide of the invention is contacted with a biological sample suspected of containing said target polypeptide. The concentration of a complex comprising the antigen-binding molecule and the target polypeptide is measure in and the measured complex concentration is then related to the concentration of target polypeptide in the sample. Consistent with the present invention, the presence of an aberrant concentration of the target polypeptide is indicative of the presence of, or probable affliction with, a cancer or tumour.

Any suitable technique for determining formation, of an antigen-binding molecule-target antigen complex may be used. For example, an antigen-binding molecule according to the invention, having a reporter molecule associated therewith may be utilized in immunoassays. Such immunoassays include, but are not limited to, radioimmunoassays (RIAs), enzyme-linked immunosorbent assays (ELISAs) and immunochromatographic techniques (ICTs), Western blotting which are well known those of skill in the art. For example, reference may be made to Coligan et al. (1994) which discloses a variety of immunoassays that may be used in accordance with the present invention. Immunoassays may include competitive assays as understood in the art or as, for example, described infra. It will be understood that the present invention encompasses qualitative and quantitative immunoassays.

Suitable immunoassay techniques are described for example in U.S. Pat. Nos. 4,016,043, 4,424,279 and 4,018,653. These include both single-site and two-site assays of the noncompetitive types, as well as the traditional competitive binding assays. These assays also include direct binding of a labelled antigen-binding molecule to a target antigen.

Two site assays are particularly favoured for use in the present invention. A number of variations of these assays exist, all of which are intended to be encompassed by the present invention. Briefly, in a typical forward assay, an unlabelled antigen-binding molecule such as an unlabelled antibody is immobilized on a solid substrate and the sample to be tested brought into contact with the bound molecule. After a suitable period of incubation, for a period of time sufficient to allow formation of an antibody-antigen complex, another antigen-binding molecule, suitably a second antibody specific to the antigen, labelled with a reporter molecule capable of producing a detectable signal is then added and incubated, allowing time sufficient for the formation of another complex of antibody-antigen-labelled antibody. Any unreacted material is washed away and the presence of the antigen is determined by observation of a signal produced by the reporter molecule. The-results may be either qualitative, by simple observation of the visible signal, or may be quantitated by comparing with a control sample containing known amounts of antigen. Variations on the forward assay include a simultaneous assay, in which both sample and labelled antibody are added simultaneously to the bound antibody. These techniques are well known to those skilled in the art, including minor variations as will be readily apparent. In accordance with the present invention, the sample is one that might contain an antigen including a tissue or fluid as described above.

In the typical forward assay, a first antibody having specificity for the antigen or antigenic parts thereof is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well known in the art and generally consist of cross-linking covalently binding or physically adsorbing, the polymer-antibody complex is washed in preparation for the test sample. An aliquot of the sample to be tested is then added to the solid phase complex and incubated for a period of time sufficient and under suitable conditions to allow binding of any antigen present to the antibody. Following, the incubation period, the antigen-antibody complex is washed and dried and incubated with a second antibody specific for a portion of the antigen. The second antibody has generally a reporter molecule associated therewith that is used to indicate the binding of the second antibody to the antigen. The amount of labelled antibody that binds, as determined by the associated reporter molecule, is proportional to the amount of antigen bound to the immobilized first antibody.

An alternative method involves immobilizing the antigen in the biological sample and then exposing the immobilized antigen to specific antibody that may or may not be labelled with a reporter molecule. Depending on the amount of target and the strength of the reporter molecule signal, a bound antigen may be detectable by direct labelling with the antibody. Alternatively, a second labelled antibody, specific to the first antibody is exposed to the target-first antibody complex to form a target-first antibody second antibody tertiary complex. The complex is detected by the signal emitted by the reporter molecule.

From the foregoing, it will be appreciated that the reporter molecule associated with the antigen-binding molecule may include the following:—

(a) direct attachment of the reporter molecule to the antigen-binding molecule;

(b) indirect attachment of the reporter molecule to the antigen-binding molecule; i.e. attachment of the reporter molecule to another assay reagent which subsequently binds to the antigen-binding molecule; and

(c) attachment to a subsequent reaction product of the antigen-binding molecule.

The reporter molecule may be selected from a group including a chromogen, a catalyst, an enzyme, a fluorochrome, a chemiluminescent molecule, a lanthanide ion such as Europium (Eu³⁴), a radioisotope and a direct visual label.

In the case of a direct visual label, use may be made of a colloidal metallic or non-metallic particle, a dye particle, an enzyme or a substrate, an organic polymer, a latex particle, a liposome, or other vesicle containing a signal producing substance and the like.

A large number of enzymes suitable for use as reporter molecules is disclosed in U.S. Pat. Nos. 4,366,241, 4,843,000 and 4,849,338. Suitable enzymes useful in the present invention include alkaline phosphatase, horseradish peroxidase, luciferase, β-galactosidase, glucose oxidase, lysozyme, malate dehydrogenase and the like. The enzymes may be used alone or in combination with a second enzyme that is in solution.

Suitable fluorochromes include, but are not limited to, fluorescein isothiocyanate (FITC), tetramethylrhodamine isothiocyanate (TRITC), R-Phycoerytbrin (RPE), and Texas Red. Other exemplary fluorochromes include those discussed by Dower et al. (International Publication WO 93/06121. Reference also may be made to the fluorochromes described in U.S. Pat. No. 5,573,909 (Singer et al), U.S. Pat. No. 5,326,692 Brinkley et al). Alternatively, reference may be made to the fluorochromes described in U.S. Pat. Nos. 5,227,487, 5,274,113, 5,405,975, 5,433,896, 5,442,045, 5,451,663, 5,453,517, 5,459,276, 5,516,864, 5,648,270 and 5,723,218.

In the case of an enzyme immunoassay, an enzyme is conjugated to the second antibody, generally by means of glutaraldehyde or periodates. As will be readily recognized, however, a wide variety of different conjugation techniques exist which are readily available to the skilled artisan. The substrates to be used with the specific enzymes are generally chosen for the production of, upon hydrolysis by the corresponding enzyme, a detectable colour change. Examples of suitable enzymes include those described supra. It is also possible to employ fluorogenic substrates, which yield a fluorescent product rather than the chromogenic substrates noted above. In all cases, the enzyme-labelled antibody is added to the first antibody-antigen complex. It is then allowed to bind, and excess reagent is washed away. A solution containing the appropriate substrate is then added to the complex of antibody-antigen-antibody. The substrate will react with the enzyme linked to the second antibody; giving a qualitative visual signal, which may be further quantitated, usually spectrophotometrically, to give an indication of the amount of antigen which was present in the sample.

Alternately, fluorescent compounds, such as fluorescein, rhodamine and the lanthanide, europium (EU), may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic colour visually detectable with a light microscope. The fluorescent-labelled antibody is allowed to bind to the first antibody-antigen complex. After washing off the unbound reagent, the remaining tertiary complex is then exposed to light of an appropriate wavelength. The fluorescence observed indicates the presence of the antigen of interest. Immunofluorometric assays (LIMA) are well-established in the art. However, other reporter molecules, such as radioisotope, chemiluminescent or bioluminescent molecules may also be employed.

It will be well understood that other means of testing the target protein (e.g. MUC13) are available, including, for instance, those involving testing for an altered level of MUC13 binding activity to a ligand or Western blot analysis of MUC13 protein levels in tissues, cells or fluids using anti-MUC13 antigen-binding molecule, or assaying the amount of antigen-binding molecule or other MUC13 binding partner which is not bound to a sample, and subtracting from the total amount of antigen-binding molecule or binding partner added.

It will of course be appreciated that discrimination between, or quantification of, the MUC13 can also be at the nucleic acid level. Further, the nucleic acid targeted can be MRNA or DNA.

Discrimination or quantification can be effected through the use of nucleotide probes. Such probes will be sufficiently complementary to part or all of the sequence of FIG. 1, to bind under stringent conditions, with the precise sequence of the probe being dependent upon which of the region of MUC13 selected to be detected. For example, nucleotide probes which target MUC13 can include a sequence complementary to that coding for the, transmembrane region or the cytoplasmic domain of MUC13.

Discrimination can also be through the use of a set of primers for amplifying nucleic acid using for example, PCR protocols such as are taught herein. For example, primers can be selected to amplify nucleic acid encoding the transmembrane domain and/or cytoplasmic domain of MUC13.

This aspect of the present invention is predicated in part on the discovery that aberrations in MUC13 or MUC13 are associated with cancers or tumours or epithelial or haematopoietic malignant or non-malignant disease conditions. Thus, the present invention contemplates a method for diagnosis in a patient of a cancer or tumour, or of the probable affliction therewith, comprising detecting an aberrant gene or aberrant expression of a gene encoding a MUC13 in a biological sample obtained from said patient.

In one embodiment, the method comprises detecting a change in the level and/or functional activity of a target molecule selected from the group consisting of an expression product of the MUC13 gene and an expression product of another gene relating to the same regulatory or biosynthetic pathway as the MUC13, gene, wherein the change is relative to a normal reference level and/or functional activity. For example, the presence of, or the probable affliction with, a cancer or tumour is diagnosed when the, MUC13 gene product is altered relative to a normal control. In a preferred embodiment of this type, the method comprises detecting a level and/or functional activity of an expression product of the MUC13 gene.

Thus, it will be desirable to qualitatively or quantitatively determine MUC13 protein levels and/or MUC13 transcription levels. Alternatively or additionally, it may be desirable to search for an aberrant MUC13 gene and/or regulatory regions. Alternatively or additionally, it may be desirable to qualitatively or quantitatively determine the level of an expression product (e.g. transcript, protein) of a gene relating to the same regulatory or biosynthetic pathway as the MUC13 gene, which can modulate or otherwise influence MUC13 protein levels and/or MUCH transcription levels. Likewise, it may also be desirable to search for an aberrant gene relating to the same regulatory or biosynthetic pathway as a MUC13 gene.

The biological sample can include any suitable tissue or fluid. Suitably, the biological sample is a tissue biopsy, preferably selected from kidney, brain, and testis.

Nucleic acid used in polynucleotide-based assays can be isolated from cells contained in the biological sample, according to standard methodologies (Sambrook, et al., “Molecular Cloning. A Laboratory Manual”; Cold Spring Harbor Press, 1989. The nucleic acid may be genomic DNA or fractionated or whole cell RNA. Where RNA is used, it may be desired, to convert the RNA to a complementary DNA. In one embodiment, the RNA is whole cell RNA; in another, it is poly-A RNA. In one embodiment, the nucleic acid is amplified by a nucleic acid amplification technique. Suitable nucleic acid, amplification techniques are well known to the skilled addressee, and include the polymerase chain reaction (PCR); strand displacement amplification (SDA) as, for example, described in U.S. Pat. No. 5,422,252; rolling circle replication RCR) as, for example, described in International Patent Publication No. WO 92/01813 and Lizardi et al. (International Application WO 97/19193); nucleic acid sequence-based amplification (NASBA) and Qβ replicase amplification.

Depending on the format, the specific nucleic acid of interest is identified in the sample directly using amplification or with a second, known nucleic acid following amplification. Next, the identified product is detected. In certain applications, the detection may be performed by visual means (e.g. ethidium bromide staining of a gel). Alternatively, the detection may involve indirect identification of the product via chemiluninescence, radioactive scintigraphy of radiolabel or fluorescent label or even via a system using electrical or thermal impulse signals (Affymax Technology).

Following detection, one may compare the results seen in a given patient with a control reaction or a statistically significant reference group of normal patients. In this way, it is possible to correlate the amount of a MUC13 detected with the progression or severity of the disease.

These defects or other aberrations in the MUC13 include deletions, insertions, point mutations and duplications. Point mutations result in stop codons, frameshift mutations or amino acid substitutions. Somatic mutations are those occurring in non-germline tissues. Germ-line tissue can occur in any tissue and are inherited. Mutations in and outside the coding region also may affect the amount of MUC13 produced, both by altering the transcription of the gene or in destabilizing or otherwise altering the processing of either the transcript (MRNA) or protein.

A variety of different assays are contemplated in this regard, including but not limited to, fluorescent in situ hybridiztion (FISH), direct DNA sequencing, pulse field gel electrophoresis (PFGE) analysis, Southern or Northern blotting, single-stranded conformation analysis,(SSCA), RNase protection assay, allele-specific oligonucleotide (ASO), dot blot analysis, denaturing gradient gel electrophoresis, RFLP and PCR-SSCP.

Primers may be provided in double-stranded or single-stranded form, although the single-stranded form is preferred. Probes, while perhaps capable of priming, are designed to bind to a target DNA or RNA and need not be used in an amplification process. In preferred embodiments, the probes or primers are labelled with radioactive species ³²P, ¹⁴C, ³⁵S, ³H, or other label), with a fluorophore (rhodamine, fluorescein) or a chemillumiscent label (luciferase).

A number of template dependent processes are available to amplify the marker sequences present in a given template sample. An exemplary nucleic acid amplification technique is the polymerase chain reaction (referred to as PCR) which is described in detail in U.S. Pat. Nos. 4,683,195, 4,683,202 and 4,800,159.

Briefly, in PCR, two primer sequences are prepared that are complementary to regions on opposite complementary strands of the marker sequence. An excess of deoxynucleoside triphosphates are added to a reaction mixture along with a DNA polymerase, e.g. Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR amplification procedure may be performed in order to quantify the amount of mRNA amplified. Methods of reverse transcribing RNA into cDNA are well known and described in Sambrook et al., 1989. Alternative methods for reverse transcription utilise thermostable, RNA-dependent DNA polymerases. These methods are described in WO 90/07641. Polymerase chain reaction methodologies are well known in the art.

Another method for amplification is the ligase chain reaction (“LCR”), disclosed in EPO No. 320 308. En LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR, bound ligated units dissociate from the target and then serve as “target sequences” for ligation of excess probe pairs. U.S. Pat. No. 4,883,750 describes a method similar to LCR for binding probe pairs to a target sequence.

Qβ Replicase described in PCT Application No. PCT/US87/0080, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′α-thio-triphosphates in one strand of a restriction site may also be useful in the amplification of nucleic acids in the present invention.

Strand Displacement Amplification (SDA) is another method of carrying out isothermal amplification of nucleic acids which involves multiple rounds of strand displacement and synthesis, i.e. nick translation. A similar method, called Repair Chain Reaction, (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5 ′ sequences of non-specific DNA and a middle sequence of specific RNA is hybridized to DNA that is present in a sample. Upon hybridization, the reaction is treated with RNase H, and the products of the probe identified as distinctive products that are released after digestion. The original template is annealed to another cycling probe and the reaction is repeated.

Still another amplification methods described in GB Application No. 2,202,328, and in PCT Application No. PCT/US89/01025, may be used in accordance with the present invention. In the former application, “modified” primers are used in a PCR-like, template- and enzyme-dependent synthesis. The primers may be modified by labelling with a capture moiety (e.g. biotin) and/or a detector moiety (e.g. enzyme). In the latter application, an excess of labelled probes are added to a sample. In the presence of the target sequence, the probe binds and is cleaved catalytically. After cleavage, the target sequence is released intact to be bound by excess probe. Cleavage of the labelled probe signals the presence of the target sequence.

Other nucleic acid amplification procedures include transcription-based amplification systems (TAS), including nucleic acid sequence based amplification NASBA) and 3SR (PCT Application WO 88/10315). In NASBA, the nucleic acids can be prepared for amplification by standard phenol/chloroform extraction, heat denaturation of a clinical sample, treatment with lysis buffer and mini-spin columns for isolation of DNA and RNA or guanidinium chloride extraction of RNA. These, amplification techniques involve annealing a primer which has target specific sequences. Following polymerisation, DNA/RNA hybrids are digested with RNase H while double stranded DNA molecules are heat denatured again. In either case the single stranded DNA is made fully double stranded by addition of second target specific primer, followed by polymerisation. The double-stranded DNA molecules are then multiply transcribed by an RNA polymerase such as T7 or SP6. In an isothermal cyclic reaction, the RNAs are reverse transcribed into single stranded DNA, which is then converted to double stranded DNA, and then transcribed once again with an RNA polymerase such as 17 or SP6. The resulting products, whether truncated or complete, indicate target specific sequences.

European Patent No. 0 329 822 disclose a nucleic acid amplification process involving cyclically synthesising single-stranded RNA (“ssRNA”), ssDNA, and double-stranded DNA (dsDNA), which may be used in accordance with the present invention. The ssRNA is a template for a first primer oligonucleotide, which is elongated by reverse transcriptase (RNA-dependent DNA polymerase). The RNA is then removed, from the resulting DNA:RNA duplex by the action of ribonuclease H (RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al. in PCT Application WO 89/06700 disclose a nucleic acid sequence amplification scheme based on the hybridisation of a promoter/primer sequence to a target single-stranded DNA (“ssDNA”) followed by transcription of many RNA copies of the sequence. This scheme is not cyclic, i.e. new templates are not produced from the resultant RNA transcripts. Other amplification methods include “RACE” and “one-sided PCR”.

Methods based on ligation of two (or more) oligonucleotides in the presence of nucleic acid having the sequence of the resulting “di-oligonucleotide”, thereby amplifying the di-oligonucleotide, may also be used in the amplification step of the present invention.

Blotting techniques are well known to those of skill in the art. Southern blotting involves the use of DNA as a target, whereas Northern blotting involves the use of RNA as a target. Each provide different types of information, although cDNA blotting is analogous, in many aspects, to blotting or RNA species.

Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic cid species followed by “blotting” on to the filter.

Subsequently, the blotted target is incubated with a probe (usually labelled) under conditions that promote denaturation and rehybridization. Because the probe is designed to base pair with the target, the probe will binding a portion of the target sequence under renaturing conditions. Unbound probe is then removed, and detection is accomplished as described above.

Products may be visualized in order to confirm amplification of the marker sequences. One typical visualisation method involves staining of a gel with ethidium, bromide and visualisation under UV light. Alternatively, if the amplification products are integrally labelled with radio- or fluorometrically-labelled nucleotides, the amplification products can then be exposed to x-ray film or visualised under die appropriate stimulating spectra, following separation.

In one embodiment, visualization is achieved indirectly. Following separation of amplification products, a labelled nucleic acid probe is brought into contact with the amplified marker sequence. The probe preferably is conjugated to a chromophore but may be radiolabelled. In another embodiment, the probe is conjugated to a binding partner, such as an antibody or biotin, and the other member of the binding pair carries a detectable moiety or reporter molecule.

In one embodiment, detection is by a labelled probe. The techniques involved are well known to those of skill in the art and can be found in many standard texts on molecular protocols. See Sambrook et al., 1989. For example, chromophore or radiolabel probes or primers identify-the target during or following amplification.

One example of the foregoing is described in U.S. Pat. No. 5,279,721, which discloses an apparatus and method for the automated electrophoresis and transfer of nucleic acids. The apparatus permits electrophoresis and blotting without external manipulation of the gel and is ideally suited to carrying out methods according to the present invention.

In addition, the amplification products described above may be subjected to sequence analysis to identify specific kinds of variations using standard sequence analysis techniques. Within certain methods, exhaustive analysis of genes is carried out by sequence analysis using primer sets designed ‘for optirnal’ sequencing. The present invention provides methods by which any or all of these types of analyses may be used. Using, for example, the sequences set forth in herein, oligonucleotide primers may be designed to permit the amplification of sequences throughout MUC13 that may then be analysed by direct sequencing.

All the essential materials and reagents required for detecting and sequencing MUC13 or MUC13 genes and variants thereof may be assembled together in a kit. The kits may also optionally include appropriate reagents for detection of labels, positive and negative controls, washing solutions, dilution buffers and the like. For example, a nucleic acid-based detection kit may include (i) a polynucleotide according to the invention (which may be used as a positive control), (ii) an oligonucleotide primer according to the invention. Also included may be enzymes suitable for amplifying nucleic acids including various polymerases (Reverse Transcriptase, Taq, Sequenase™ DNA ligase etc. depending on the nucleic acid amplification technique employed), deoxynucleotides and buffers to provide the necessary reaction mixture for amplification. Such kits also generally will comprise, in suitable means, distinct containers for each individual reagent and enzyme as well as for each primer or probe.

Also contemplated by the present invention are chip-based DNA technologies. Briefly, these techniques involve quantitative methods for analysing large numbers of genes rapidly and accurately. By tagging genes with oligonucleotides or using fixed probe arrays, one can employ chip technology to segregate target molecules as high density arrays, and screen these molecules on the basis of hybridization.

Thus, in accordance with the invention there is provided a new tm-mucin, MUC13.

The applications of MUC13 are numerous. One application is in the identification of ligands which bind MUC13. Such ligands can either be stimulatory ligands in that they bind to and activate MUC13 or inhibitory, in that they bind to but do not activate MUC13.

Ligands which can be screened for may bind to the extracellular domain of MUC13 or the cytoplasmic domain of MUC13.

The design and implementation of a screening assay by which such ligands can be identified and characterized will be routine to those persons skilled in the art. By way of example, a polynucleotide encoding MUC13 can be incorporated into cell lines (such as chinese hamster ovary (CHO) cell s) where the expressed protein is capable of producing a biological response or capable of binding potential ligands that are added.

The skilled worker will also recognise that it will be possible to produce antibodies, particularly monoclonal antibodies, which are capable of functioning as stimulatory or inhibitory ligands. Such antibodies can be produced as described above.

Such ligands have application in the modulation of MUC13 function. Such modulation may involve either stimulation or inhibition of MUC13 function.

Inhibition of MUC13 function may also be achieved with a soluble form of the extracellular domain of MUC13, or a fragment of that domain to which a circulating stimulatory ligand binds. Such a soluble protein can be prepared using the same techniques as for MUC13 itself.

The antibodies of the invention have application in prognostic or diagnostic protocols. By way of example, the antibodies, optionally labelled, can be employed to detect MUC13 in respiratory mucus and/or tissues from individuals with respiratory conditions for the purpose of predicting disease severity and/or prognosis and/or responsiveness to treatment. Similarly, the antibodies, optionally labelled, can be employed to detect MUC13 in the serum of patients with cancers of epithelial origin, or in patients with other epithelial or haematopoietic malignant or non-malignant conditions in which MUC13 is found in the serum. Similarly, antibodies reactive with MUC13 can be used to define specific haematopoietic cell populations based, on cell surface or intracellular expression of MUC13.

It is equally practical to employ nucleotide probes or primers as described above in such applications.

Antibodies which target MUC13 could also form part of therapeutics against diseases or conditions which involve over-expression of MUC13. In such therapeutics, the antibody component can be used coupled to a toxin to deliver the toxin to the diseased cells.

Antibodies reactive with cell surface MUC13 on haematopoietic cells could be used to either deplete MUC13 expressing cells from haematopoietic cell populations, or to purify, the MUC13 expressing cells. For example, MUC13 expressing, bone marrow or peripheral blood haematopoietic precursor cells could be enriched or depleted from preparations of precursor cells for the purpose of transplantation.

The inventors also propose that alterations in DNA encoding MUC13 will be representative of a predisposition to epithelial or haematopoietic malignant or nonmalignant disease as well as for providing prognostic or predictive information relating to the outcome, severity or responsiveness to treatment of a patient suffering from such a disease. Such alterations can be identified using antibodies as defined above or, more usually, by screening protocols performed at the nucleic acid level.

As discussed above, “alteration of a MUC13 gene” encompasses all forms of mutations including deletions, insertions, point mutations and VNTR polymorphisms in the coding and noncoding regions. Point mutations may result in stop codons, frameshift mutations or amino acid substitutions.

Detection of point mutations may be accomplished by molecular cloning of the MUC13 allele(s) and sequencing that allele(s) using techniques well known in the art and/or as described herein.

MUC13 sequences generated by amplification may be sequenced directly. Alternatively, the amplified sequence(s) may be cloned prior to sequence analysis. A method for the direct cloning and sequence analysis of enzymatically amplified genomic segments has been described by Scharf, 1986.

There are numerous well known methods for confirming the presence of a susceptibility allele. These include: (1) single stranded confirmation analysis (“SSCA”); (2) denaturing gradient gel electrophoresis (“DGGE”); (3) RNase protection assays; (4) allele-specific oligonucleotides' (ASO's); (5) the use of proteins which recognize nucleotide mismatches, such as the E. coli mutS protein; and (6) allele-specific PCR. For allele-specific PCR, primers are used which hybridize at their 3′ ends to a particular mutation. If the particular mutation is not present, an amplification product is not observed.

Other approaches which can also be used include the Amplification Refractory Mutation System (ARMS), as disclosed in European Patent Application Publication No. 0 332 435.

In similar fashion, DNA probes can be used to detect mismatches, through enzymatic or chemical cleavage. Alternatively, mismatches can be detected by shifts in the electrophoretic mobility of mismatched duplexes relative to matched duplexes. With either riboprobes or DNA probes, the cellular mRNA or DNA which might contain a mutation can be amplified using PCR before hybridization. Changes in DNA of the MUC13 gene can also be detected using Southern hybridizations especially if the changes are gross rearrangements, such as deletions and insertions.

Mutations from potentially susceptible patients falling outside the coding-region of MUC13 can be detected by examining the non-coding regions, such as introns and regulatory sequences near or within the MUC13 gene. An early indication that mutations in noncoding regions are important may come from Northern blot experiments that reveal messenger RNA molecules of abnormal size or abundance in patients as compared to control individuals.

Antibodies specific for products of an altered MUC13 gene could also be used to detect mutant MUC13 gene product. Such antibodies can be produced in equivalent fashion to the antibodies for MUC13 as described above.

Early at-risk determination provides the opportunity for early intervention. Carriers of the mutation could choose to have prophylactic treatment.

There is also the possibility of a curative or corrective approach using gene therapy. This will involve supplying wild-type MUC13 function to an individual who carries an altered MUC13 gene. The wild-type gene or a part of the gene may be introduced into-cells within such an individual in a vector such that the gene remains extrachromosomal. In such a situation, the gene will be expressed by the cell from the extrachromosomal location. If a gene portion is introduced and expressed in a cell carrying a mutant allele, the gene portion should encode a part of the protein which is required for non-neoplastic growth of the cell. More usual is the situation where the wild-type gene or a part thereof is introduced into the mutant cell in such a way that it recombines with the endogenous mutant gene present in the cell. Such recombination requires a double recombination event which results in the correction of the gene mutation. Vectors, for introduction of genes both for recombination and for extrachromosomal maintenance are known in the art, and any suitable vector may be used. Methods for introducing DNA into cells such as electroporation calcium phosphate co-precipitation and viral transduction are known in the art.

The present invention is further described by the following non-limiting Examples.

### EXAMPLE

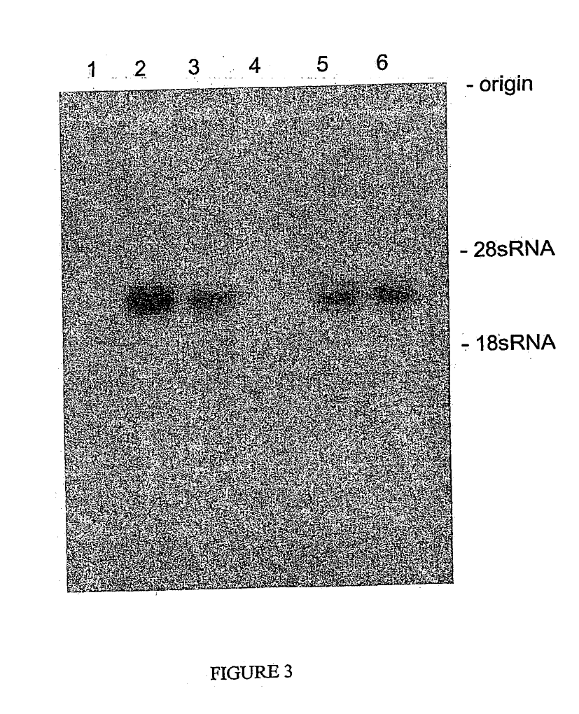

The invention and its application is, in part, represented in the accompanying drawings, and in particular FIGS. 3 to 13, with FIGS. 1 and 2 giving the nucleotide and amino acid sequences for MUC13, respectively. The MUC13 sequence is deposited in GenBank on 9 Jul. 2000 under Accession No. AF286113. [gi/14209831/gb/AF286113.1/AF286113 [14209831].

Analysis of the amino acid sequence of FIG. 2 reveals a series of serine/threonine rich very degenerate tandem repeats that lead into an EGF-like domain, followed by a domain containing a SEA module, then two further adjacent EGF-like domains, a hydrophobic transmembrane region and a 69 amino acid cytoplasmic tail.

FIG. 3 shows a Northern blot analysis of the MUC13 MRNA Total RNA was isolated using Trizol (Gibco) from three colonic cancer, two breast cancer and one pancreatic cancer cell lines and subjected to electrophoresis in a 1% w/v agarose gel containing 0.66 M formaldehyde and transferred to a nylon membrane (Hybond N⁺ (Amersham). A CDNA probe corresponding to nucleotides 688-1770 in FIG. 1 was labelled with 32P, hybridized to the membrane, and washed with high stringency, and exposed to X-ray film for 18 h. A single band of approximately 3100 bp in size was observed in all colon cancer cell lines and the pancreatic cancer cell line, and no signal was seen in the breast cancer cell lines. Lane 1=MA11 breast cancer cell line, 2=HT29 colon cancer cell line, 3=CAPAN-1 pancreatic cancer cell line, 4 MDA-MB-453 breast cancer cell line, 5=CACO-2 colon cancer cell line, 6=SW620 colon cancer cell line.

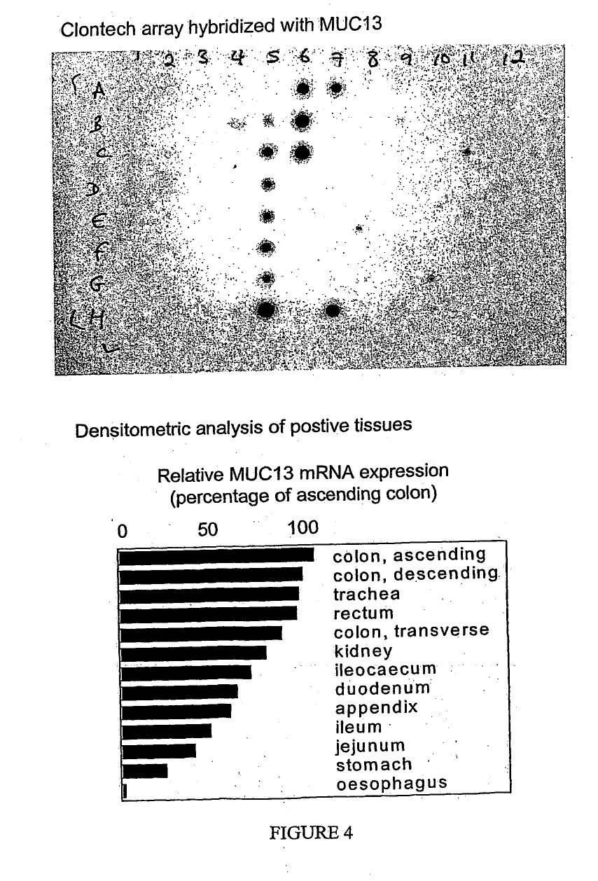

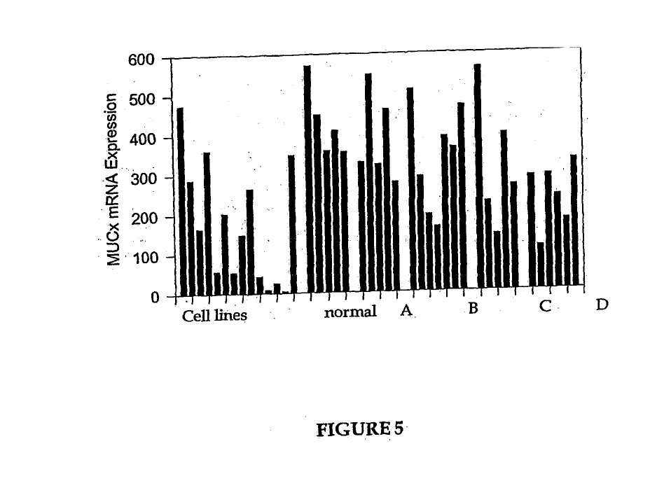

FIG. 4 shows expression of MUC13 mRNA in 79 normal human tissues. Hybridization was performed as in FIG. 3 using a commercial RNA array (Clontech Cat. 7775-1).

FIG. 5 shows expression of MUC13 mRNA in normal tissues and cancers of the colon. Total RNA was isolated from colorectal cancer cell lines and surgical specimens of normal colon and colonic cancers of Duke's stages A, B, C and D. Integrity of the RNA was confirmed by assessing the integrity of ribosomal bands under denaturing electrophoresis. Twenty ug of RNA was denatured in formaldehyde and formamide and applied to a nylon membrane (Hybond N⁺ (Amersham) using a 96-well dot blot apparatus [Biorad]. A MUC13 cDNA probe was hybridized to the blots as described in FIG. 3. Densitometry was used to evaluate hybridization and each result was expressed as a percentage of a control sample. All samples shown were hybridized on a single blot.

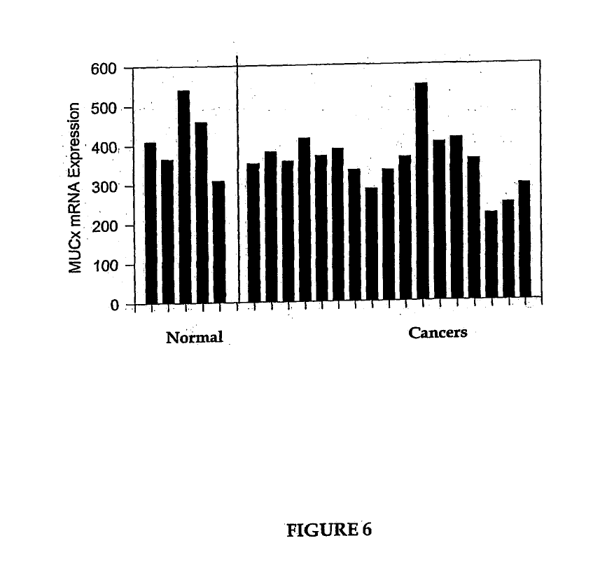

FIG. 6 shows expression of MUC13 mRNA in normal tissues and cancers of the rectum. Total RNA was isolated from surgical specimens of normal rectum and rectal cancers. Integrity of the RNA was confirmed by assessing the integrity of ribosomal bands under denaturing electrophoresis. Twenty ug of RNA was denatured in formaldehyde and formamide and applied to a nylon membrane (Hybond N⁺ (Amersham) using a 96-well dot blot apparatus [Biorad]. A MUC13 cDNA probe was hybridized to the blots as described for FIG. 3. Densitometry was used to evaluate hybridization and, each result was expressed as a percentage of a control sample. All samples shown were hybridized on a single blot.

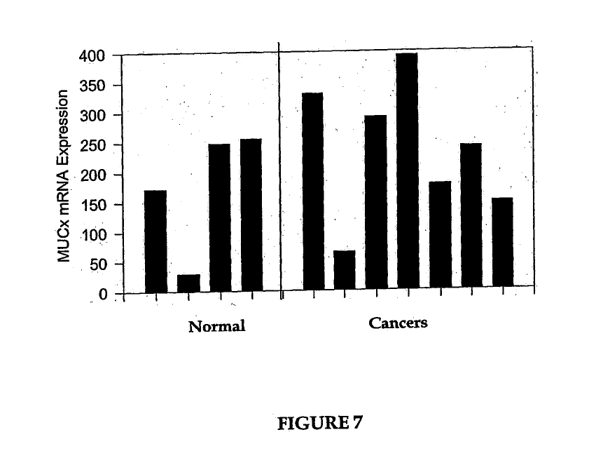

FIG. 7 shows expression of MUC13 MRNA in normal tissues and cancers of the stomach. Total RNA was isolated from surgical specimens of normal stomach and gastric, cancers. Integrity of the RNA was confirmed by assessing the integrity of ribosomal bands under denaturing electrophoresis. Twenty ug of RNA was denatured in formaldehyde and formamide and applied to a nylon membrane (Hybond NE (Amersham) using a 96-well dot blot apparatus [Biorad]. A MUC13 cDNA probe was hybridized to the blots as described for FIG. 3. Densitometry was used to evaluate hybridization and each result was expressed as a percentage of a control sample. All samples shown were hybridized on a single blot.

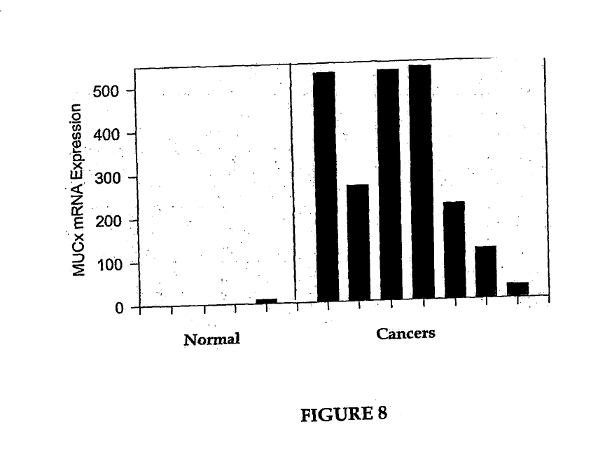

FIG. 8 shows expression of MUC13 mRNA in normal tissues and cancers of the oesophagus. Total RNA was isolated from surgical specimens of normal oesophagus and oesophageal cancers. Integrity of the RNA was confirmed by assessing the integrity of ribosomal bands under denaturing electrophoresis. Twenty ug of RNA was denatured in formaldehyde and formamide and applied to a nylon membrane (Hybond N⁺ (Amersham) using a 96-well dot blot apparatus [Biorad]. A MUC13 cDNA probe was, hybridized to the blots as described for FIG. 3. Densitometry was used to evaluate hybridization and each result was expressed as a percentage of a control sample. All samples shown were hybridized on a single blot.

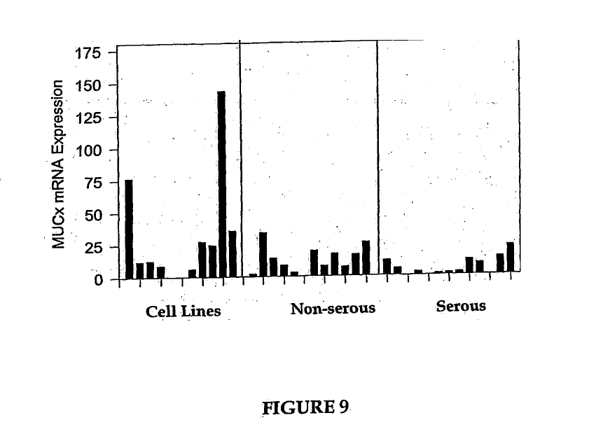

FIG. 9 shows expression of MUC13 mRNA in normal tissues and cancers of the ovary. Total RNA was isolated from ovarian cancer cell lines and surgical specimens of serous and non-serous ovarian cancers. Integrity of the RNA was confirmed by assessing the integrity of ribosomal bands under denaturing electrophoresis. Twenty ug of RNA was denatured in formaldehyde and formamide and applied to a nylon membrane (Hybond N⁺ (Amersham) using a 96-well dot blot apparatus [Biorad]. A MUC13 cDNA probe was hybridized to the blots as described for FIG. 3. Densitometry was used to evaluate, hybridization and each result was expressed as a percentage of a control sample. All samples shown were hybridized on a single blot.

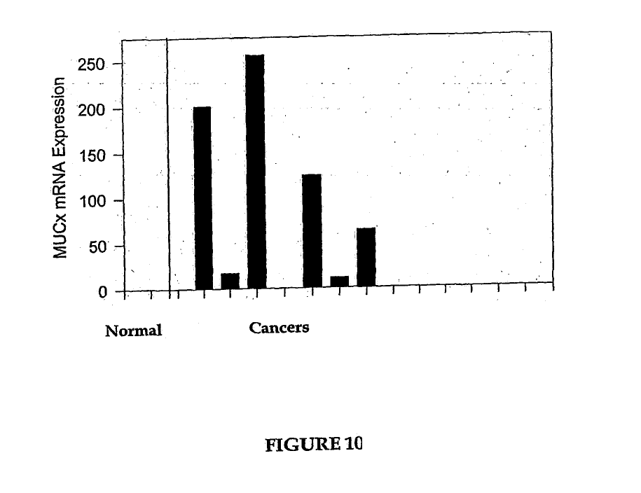

FIG. 10 shows expression of MUC13 mRNA in normal tissues and cancers of the bladder. Total RNA was isolated from surgical specimens of normal bladder and bladder cancers. Integrity of the RNA was confirmed by assessing the integrity of ribosomal bands under denaturing electrophoresis. Twenty ug of RNA was denatured in formaldehyde and formamide and applied to a nylon membrane (Hybond N⁺ (Amersham) using a 96-well dot blot, apparatus [Biorad]. A MUC13 cDNA probe was hybridized to the blots as described for FIG. 3. Densitometry, was used to evaluate hybridization and each result was expressed as a percentage of a control sample. All samples shown were hybridized on a single blot.

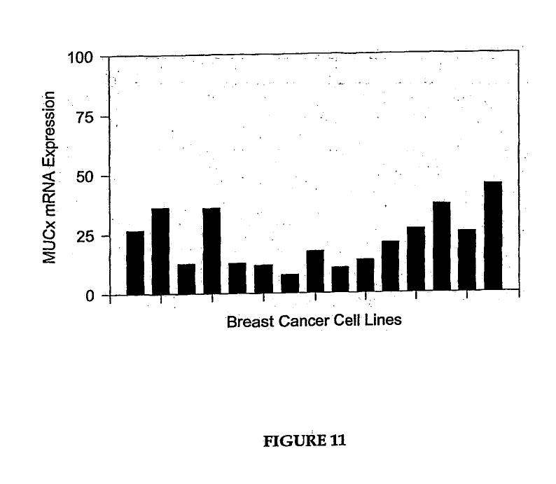

FIG. 11 shows expression of MUC13 mRNA in breast cancers. Total RNA was isolated from breast cancer cell lines. Integrity of the RNA was confirmed by assessing the integrity of ribosomal bands under denaturing electrophoresis. Twenty, ug of RNA was denatured in formaldehyde and formamide and applied to a nylon membrane (Hybond N⁺ (Amersham) using a 96-well dot blot apparatus [Bio'rad]. A MUC13 CDNA probe was hybridized to the blots as described for FIG. 3. Densitometry was used to evaluate hybridization and each result was expressed as a percentage of a control sample. All samples shown were hybridized on a single blot.

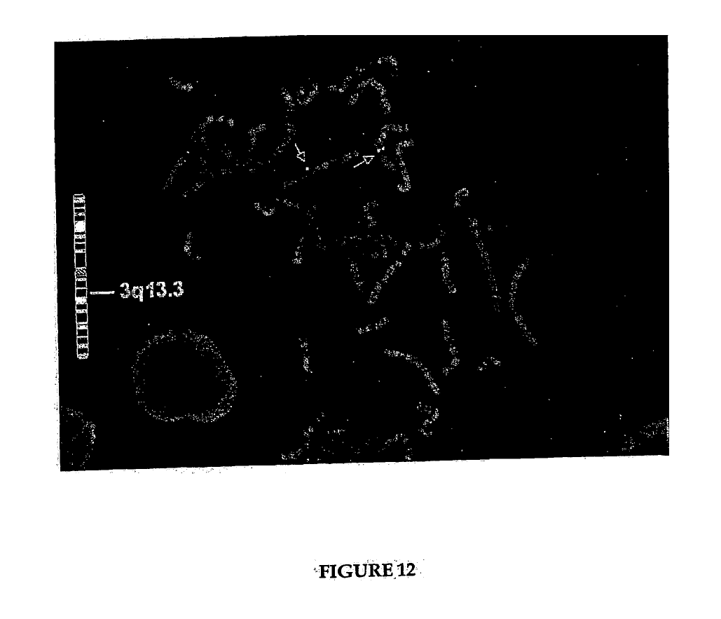

FIG. 12 shows the results of Fluorescence in situ hybridization. MUC13 (688-1770 FIG. 1) was nick translated with biotin-14-dATP and hybridized in situ at a final concentration of 10 ng/μl to metaphases from two normal males. The fluorescence in situ hybridization (FISH) method was modified from that previously described, in that chromosomes were stained before analysis with both propidium iodide as counterstain and DAPI for chromosome identification. Images of metaphase preparations were captured by a cooled CCD camera using the Cyro Vision Ultra image collection and enhancement system (Applied Imaging Int Ltd, Sunderland, UK.)

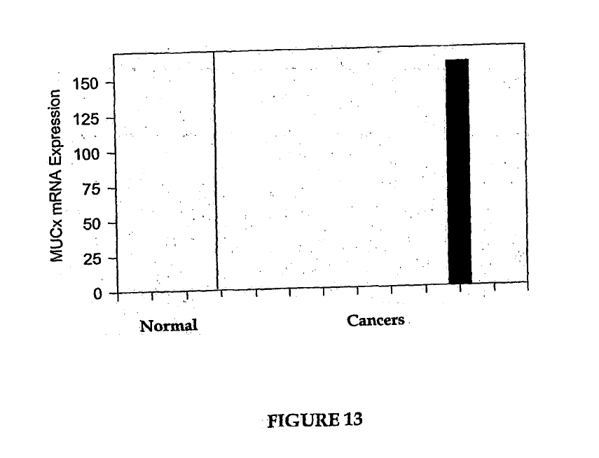

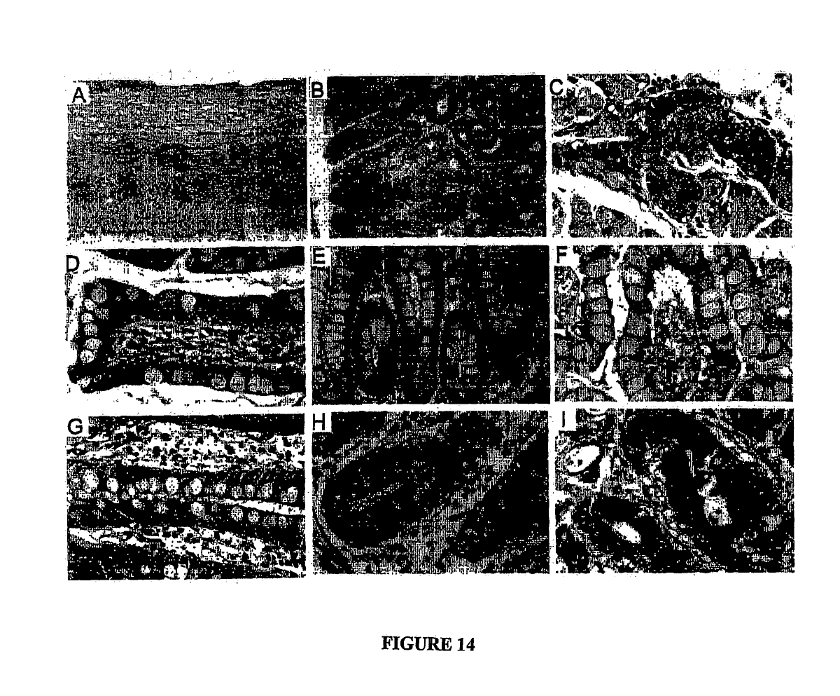

FIG. 13 shows expression of MUC13 mRNA in normal tissues and cancers of the kidney. Total RNA was isolated from surgical specimens of normal kidney and renal cell carcinomas. Integrity of the RNA was confirmed by assessing the integrity of ribosomal bands under denaturing electrophoresis. Twenty ug of RNA was denatured in formaldehyde and formamide and applied to a nylon membrane (Hybond N⁺ (Amersham) using a 96-well dot blot apparatus [Biorad]. A MUC13 cDNA probe was hybridized to the blots as described for FIG. 3. Densitometry was used to evaluate hybridization and each result was expressed as a percentage of a control sample. All samples shown were hybridized on a single blot

FIG. 14 shows immunohistochemical detection of human MUC13 protein in epithelial tissues. Three synthetic peptides were synthesized (Auspep, Parkville, Australia) corresponding to three hydrophilic sequences of the putative MUC13 amino acid sequence, each with an terminal cysteine residue added: an extracellular domain epitope (peptide A, DPEEKHSMAYQDLHSEC, amino acids 229-244 in FIG. 2) and two cytoplasmic tail epitopes (peptide B, CRSNNTKHIEEENLID amino acids 446-461 in FIG. 2 and peptide C, CMQNPYSRHSSMPRPDY amino acids 497-512 in FIG. 2). These peptides were conjugated to bovine serum albumin (BSA) using glutaraldehyde. Six week old female Balb/c mice were immunized intra-peritoneally with 25 μg of conjugated peptide initially in 0.2 mL complete Freund's adjuvant:PBS 1:1 (Life Technologies), and then at three-weekly intervals in incomplete Freund's adjuvant. Blood samples were obtained via cardiac puncture under terminal anaesthesia, clotted, and serum stored at −20° C. Reactivity of serum with the peptides was assessed using the specific and irrelevant unconjugated peptides as solid phase in an ELISA. Polyclonal MUC13 peptide-reactive mouse sera were used to detect MUC13 in paraffin sections of normal and diseased human gastrointestinal epithelial tissues. Immunohistochemistical techniques were as previously described (Walsh et al., 1999) with peptide A and B reactive sera diluted 1/100 and tissue sections subject to antigen retrieval by boiling in 0.1 M citric acid pH 6, and peptide C reactive sera diluted 1/400 with antigen retrieval not used. Polyclonal antisera raised against peptides A, B and C reacted similarly with paraffin sections of fixed human gastrointestinal tissues with the peptide C-reactive antisera showing the best reactivity and not requiring antigen retrieval of sections. Surprisingly, given the lack of reactivity on the human mRNA master blot, intense MUC13 staining was observed in the cytoplasm of squamous epithelial cells of the esophagus, with staining absent or very weak in the basal cell layer (see FIG. 14A). In the body of the stomach, MUC13 was expressed on the apical membrane surface of cells of the surface epithelium, the gastric pits, and the more peripheral glands but only occasionally in the deep glands (see FIG. 14B). In addition, in the gastric glands occasional mucus, neck cells showed intense granular cytoplasmic, staining (see FIG. 14B). Some mucus cells of the surface epithelium and gastric pits also showed moderate cytoplasmic reactivity, and in some cells supranuclear staining was also observed. In the pyloric and cardiac stomach some of the deep glands showed moderate to strong cytoplasmic expression of MUC13, whilst adjacent glands were often negative (FIG. 14C). In the duodenum, MUC13 was detected in supranuclear vacuoles within all absorptive cells, consistent with detection in the Golgi region, however, apical membrane staining was not seen (see FIG. 14D). MUC13 was also expressed on the apical membrane of epithelial cells lining pancreatic ducts. In the terminal ileum of the small intestine, MUC13 was detected as intense staining of the apical membrane of all cells deep in the crypts (see FIG. 14E) and less frequent apical membrane staining of cells of the surface epithelium (see FIG. 14F). Both goblet and columnar cells appeared to express cell surface MUC13, however, staining was more intense in columnar cells and at high power a microvillous-type pattern was observed. Secreted material in the crypt lumen also stained. In addition, moderate to weak staining in mesh network and punctate patterns was also seen within the thecae of goblet cells in the villi (see FIG. 14F) but only rarely deep in the crypts (see FIG. 14E). The staining of goblet cell thecae was more pronounced using peptide A-reactive antisera than C-reactive antisera (not shown). Appendix showed strong MUC13 immunoreactivity both in the cytoplasm and on the cell surface of both goblet and columnar cells, with very strong cytoplasmic reactivity in columnar cells (see FIG. 14G). In the colon, MUC13 was, similarly to the terminal ileum, highly expressed on the apical membrane surface of both columnar and goblet cells deep in the crypts (see FIG. 14H). In addition, supranuclear vacuolar staining, like that seen in the duodenum, also observed in these cells together with reactivity with secreted material. Occasional columnar cell apical membrane staining was observed on the colonic surface epithelium together with goblet cell thecal staining, although this was less intense than that seen in the terminal ileum. In a small series of colorectal cancers examined, MUC13 was expressed in the cytoplasm and on the cell surface of cancer cells, however, expression of MUC13 was often low relative to normal tissue and was typically heterogeneous in nature (see FIG. 14I).

The above figures allow a number of conclusions to be drawn, particularly relating to the amino acid sequence of FIG. 2. This sequence shows numerous serine and threonine residues which are potential sites for O-linked glycosylation, particularly in the N-terminal mucin repeat domain. Three potential N-glycosylation sites are present in the region flanked by and including the EGF-like domains. The presence of a SEA module makes it likely that the MUC13 protein is cleaved during synthesis in the endoplasmic reticulum and that the extracellular subunit containing the first EGF-like domain and the mucin repeat domain could be shed from the cell surface. Interestingly, the cytoplasmic domain contains a signal sequence for direction into clathrin-coated vesicles. This suggests that MUC13 can be internalized by clathrin-mediated mechanisms implicating it as a cargo-carrier in endocytic pathways. Furthermore, the cytoplasmic domain of MUC13 includes two tyrosine residues and seven serine residues which represent potential phosphorylation sites and may mediate signal transduction by this molecule.

The results above also show high levels of MUC13 expression in a broad range of human epithelial cancers, particularly in cancers of the intestinal tract. Substantial amounts of MUC13 may therefore be shed into the serum of patients with these cancers, providing the opportunity to test for the presence of MUC13 in serum as a diagnostic-or prognostic tool. In addition, the above results show differential expression of MUC13 as between normal and cancerous tissues throughout the body. Such differential expression gives rise, inter alia, to the opportunity to test for MUC13 expression as a diagnostic or prognostic tool.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

Bibliography

Seregni, E., Botti, C., Massaron, S., Lombardo, C., Capobianco, A., Bogni, A., Bombardieri E. (1997) Tumori, 83, 625-632.

Gendler, S. J., Spicer, A. P., Lalaini, E. N., Duhig, T., Peat N., Burchell, J., Pemberton, L., Boshell, M., Taylor-Papadimitniou, J. (1991) Am. Rev. Respir. Dis., 144, S42-S47.

Sheng, S., Wu, K, Carraway, K. L. & Fregien, N. (1992) J. Biol. Chem., 267, 16341-16346.

Moniaux, N., Nollet, S., Porchet, N., Degand, P., Laine, A., Aubert, J. P. (1999) Biochem. J. 338, 325-333.

Campbell, I. D., Bork, P. (1993) Epidermal growth factor-like modules. Curr. Opin. Struct. Biol., 3, 385-392.

Mcneer, R. R., Price-Schiavi, S. A., Komatsu, M., Fregien, N., Carraway, C. A. C. & Carraway, K. L. (1998) Front. Bioscience, 2, 449-459.

Gum, J. R., Ho, J. J. L., Pratt, W. S., Hicks, J. W., Hill, A. S., Vinall, L. E., Roberton, A. M., Swallow, D. M., Kim, Y. S. (1997) J. Biol. Chem., 272, 26678-26686.

Williams, S. J., McGuckin, M. A., Gotley, D. J., Eyre, H.; Sutherland, G., Antalis, T. M. (1998). Cancer Res. (in press).

Chomczynski, P., Sacchi, N. (1987) Anal Biochem., 162, 156-159.

Don, R. H., Cox, P. T., Wainwright, B. J., Baker, K., Mattick, J. S. (1991) N.A.R., 19, 4008.

Altschul, S. F., Gish, W., Miller, W., Myers, E. W., Lipman, D. J. (1990) J. Mol. Biol., 215, 403-410.

Songyang, Z., Shoelson, S. E., McGlade, J., Olivier, P., Pawson, T., Bustello, X. R., Barbacid, M., Sabe, H., Hanafusa, H., Yi, T., Ren, R., Baltimore, D., Ratnofsky, S., Feldman, R. A. & Cantley, L. C. (1994) Mol. Cell Biol., 14, 2777-2785.

Zrihan-Licht, S., Baruch, A., Elroy-Stein, O., Keydar, I., Wreschner, D. H. (1994) FEBS Lett., 356, 130-136.

Wreschner, D. H., Hareuvini, M., Tsarfaty, I., Smorodinsky, N., Horev, J., Zaretsky, J., Kotkes, P., Weiss, M., Lathe, R., Dion, A. S., Keydar, I. (1990) Eur J. Biochem., 189, 463-473.

Zrihan-Licht, S., Vos, H. L., Baruch, A., Elroy-Stein, O., Sagiv, D., Keydar, I., Hilkens, J., Wreschner, D. H. (1994) Eur. J. Cancer, 224, 787-795.

Gum, J. R., Hicks, J. W., Lagace, R. L., Byrd, J. C., Toribara, N. W., Siddiki, B., Fearney, F. J., Lamport, D. T., Kim, Y. S. (1991) J. Biol., Chem., 266, 22733-22738.

Khatri, I. A., Forstner, G. G., Forstner, J. F. (1997) Biochim. Biophys. Acta., 1326, 7-11.

Shekels, L. L., Hunninghake, D. A., Tisdale, A. S., Gipson, I. K., Kieliszewski, M., Kozak, C. A, Ho, S. B. (1998) Biochem. J., 330, 1-301-1308.

Ogata, S., Uehara, H., Chen, A., Itzkowitz, S. H. (1992) Cancer Res, 52, 5971-5978.

Chang, S., Dohrman, A. F., Basbaum, C. B., Ho, S. B., Tsuda, T., Toribara, N. W., Gum, J. R., Kim, Y. S. (1994) Gastroenterology, 107, 28-26.

Satsangi, J., Parkes, M., Louis, E., Hashimoto, L., Kato, N., Welsh, K., Terwilliger, J. D., Lathrop, G. M., Bell, J. I., Jewell, D. P. (1996) Nature Genet., 14, 199-202.

Kyo, K., Parkes, M., Takei, Y., Nishimori, H., Vyas, P., Satsangi, J., Simmons, J., Nagawa, H., Baba, S., Jewell, D., Muto, T., Lathrop, M., Nakamura Y. (1999) Hum. Mol. Gen, 8, 307-311.

Rhodes, J. M. (1997) Q. J. Med. 90, 79-82.

Parmley, R. R. and Gendler, S. J. (1998) J. Clin. Invest. 102, 1798-1806.

Altschul, S. F., et al. (1997) Nucleic Acids Res. 25: 3389-3402.

Harlow & Lane (1988) Antibodies: A Laboratory Manual, Cold Spring Harbour Press, 144.

Hodgson J. (1991) Making Monoclonals in Microbes, Biotechnology 9:423-425.

Cariello (1988). Human Genetics 42:726.

Conner, B. J., et al. (1983). Proc. Nat. Acad Sci USA. 80:278-282.

Finkelstein, J., et al. (1990). Genomics 7:167-172.

Innis et al., (1990). PCR Protocols: A Guide to Methods and Applications, (Academic Press, San Diego).

Kinszler, K. W., et al., (1991). Science 251:1366-1370.

Modrich, P. (1991). Ann. Rev. Genet. 25:229-253.

Newton, C. R., Graham, A., Heptinstall, L. E., Powell, S. J., Summers, C., Kalsheker, N., Smith, J. C., and Markham, A. F. (1989). Nucl. Acids Res. 17:2503-2516.

Novack, et al., (1986). Proc. Nat. Acad. Sci. USA 83:586.

Orita, et al., (1989). Proc. Nat. Acad. Sci. USA 86:276-2770.

Orita, et al., (1989). Genomics 5:874-879.

Rano & Kidd (1989). Nucl. Acids Res. 17:8392.

Scharf (186). Science 233:1076.

Sheffield, V. C., et al., (1989). Proc. Nat. Acad. Sci. USA 86:232-236.

Devine, P. L. & McGuckin, M. A. (1994) The cancer-associated serum antigen (CASA) in the management of patients with carcinoma (Review). Aust. J. Med. Sci. 15, 126-132.

McGuckin, M. A. & Devine, P. L. (1995) The mucin marker, CASA, in ovarian cancer (Review). Tumor Diagnostik and Therapie 16, 1-6.

Ohgami, A., Tsuda, T., Osaki, T., Mitsudomi, T., Morimoto, Y., Higashi, T., and Yasumoto, K (1999) MUC1 mucin mRNA expression in stage I lung adenocarcinoma and its association with early recurrence. Ann. Thor Surg. 67, 810-814.

Fujita, K., Denda, K., Yamamoto, M., Matsumoto, T., Fujime, M., and Irimura, T. (1999) Expression of MUC1 mucins inversely correlated with post-surgical survival of renal cell carcinoma patients. Br. J. Cancer 80, 301-308. Hiraga, Y., Tanaka, S., Haruma, K., Yoshihara, M., Sumii, K., Kajiyama, G., Shimamoto, F., and Kohno, N. (1998) Immunoreactive MUC1 expression at the deepest invasive portion correlates with prognosis of colorectal cancer. Oncology 55, 307-319.

McGuckin, M. A., Walsh, M. D., Hohn, B. G., Ward, B. G. & Wright, R. G. (1995) Prognostic significance of MUC1 epithelial mucin expression in carcinoma of the breast. Human Pathol. 26, 432-439.

Agrawal, B., Gendler, S. J., and Longenecker, B. M. (1998) The biological role of mucins in cellular interactions and immune regulation: prospects for cancer immunotherapy [review]. Molecular Medicine Today 4, 397-403.

McGuckin, M. A., MacDonald, K. P. A., Tran, M., Wykes, M. and Hart, D. N. J. (2000) MUC1 Epithelial Mucin—Expression by Normal Haematopoietic Cells. Leukocyte Typing VII, Ed. D. Mason (in press).

Takahashi, T., Makiguchi, Y., Hinoda, Y., Kakiuchi, H., Nakagawa, N., Imai, K., and Yachi, A. Expression of MUC1 on myeloma cells and induction of HLA-unrestricted CTL against MUC1 from a multiple myeloma patient. J. Immunol. 153, 2102-2109 (1994).

Treon, S. P., Mollick, J. A., Urashima, M., Teoh, G., Chauhan, D., Ogata, A.; Raje, N., Hilgers, J. H. M., Nadler, L., Belch, A. R., Pilarski, L. M., and Anderson, K. C. Muc-1 core protein is expressed on multiple myeloma cells and is induced by dexamethasone. Blood 93, 1287-1298 (1999).

Brugger, W., Buhring, H. J., Grunebach, F., Vogel, W., Kaul, S., Muller, R., Brummendorf, T. H., Ziegler, B. L., Rappold, I., Brossart, P., Scheding, S., and Kanz, L. Expression of MUC-1 epitopes on normal bone marrow: implications for the detection of micrometastatic tumor cells. J. Clin. Oncol. 17, 1535-1544 (1999).

Walsh, M. D., Luckie, S. M., Cummings, M. C., Antalis, T. M. and McGuckin, M. A., Breast Cancer Res. Treat. 58: 255-266 (1999).

1. An isolated MUC13 protein comprising an amino acid sequence substantially as set forth in SEQ ID NO:1 or a protein which is functionally equivalent or a variant thereof and/or a protein which comprises an amino acid sequence having at least about-60% similarity to SEQ ID NO:1 or a homologue, derivative or chemical analogue of said protein.

2. An isolated MUC13 protein according to claim 1 wherein the protein is encoded by a nucleic acid molecule comprising a nucleotide sequence substantially as set forth in SEQ ID NO:2 or a nucleotide sequence having at least about 60% similarity to SEQ ID NO:2 after optimal alignment or a nucleotide sequence capably of hybridising to SEQ ID NO:2 or its complementary form under low stringency conditions.

3. An isolated MUC13 protein according to claim 1 or 2 derived from a human.

4. An isolated MUC13 protein according to, claim 1 or 2 or when present in a composition comprising one or more pharmaceutically acceptable diluents and/or carriers.

5. An isolated MUC13 protein according to claim 1 wherein the MUC13 amino acid sequence is as set forth in SEQ ID NO:1.

6. A derivative of the MUC13 protein as defined in claim 5.

7. An isolated nucleic acid molecule comprising a sequence of nucleotides which encodes an amino acid sequence as set forth in SEQ ID NO:1 or an amino acid sequence having at least about 60% similarity thereto.

8. An isolated nucleic acid molecule according t6 claim 7 comprising a nucleotide sequence substantially as set forth in SEQ ID NO:2 or a nucleotide sequence having at least about 60% similarity to SEQ ID NO:2 after optimal alignment or a nucleotide sequence capably of hybridising to SEQ ID NO:2 or its complementary form under low stringency conditions.

9. An isolated nucleic acid molecule according to claim 7 or 8 comprising the nucleotide sequence substantially set forth in SEQ ID NO:2.

10. A derivative of the nucleic acid molecule according to claim 9.

11. An antibody capable of interacting with the protein defined in any one of claims 1 to 6.

12. An antibody according to claim 11 wherein the antibody is a monoclonal antibody or an antigen binding fragment thereof.

13. An antibody according to claim 11 wherein the antibody is a polyclonal antibody or an antigen binding fragment thereof.

14. An antibody according to claim 11 or 12 or 13 wherein the antibody does not substantially interact with the proteins MUC1, MUC3, MUC4 or MUC12.

15. A primer or probe comprising a nucleic acid molecule sufficiently complementary with the polynucleotide sequence defined in SEQ ID NO:2 to bind under low stringency conditions.

16. A method for detecting MUC13 protein in a biological sample said method comprising contacting said biological sample with a MUC13 specific antibody for a time and under conditions sufficient for MUC13-antibody complex to form and then the detecting the presence of said complex.

17. A method according to claim 16 wherein the MUC13 specific antibody is labeled with a reporter molecule.

18. A method according to, claim 16 wherein the MUC13-antibody complex is detected by anti-immunoglobulin antibody labeled with a reporter molecule.

19. A method for detecting an aberrant MUC13 protein in a biological sample said method comprising contacting said sample with a MUC13 specific antibody for a time under conditions sufficient for a MUC13-antibody complex to form wherein the absence of said complex is indicative of an aberrant MUC13.

20. A method according to claim 19 wherein the MUC13 specific antibody is labeled with a reporter molecule.

21. A method according to claim 19 wherein the MUC13-antibody complex is detected by anti-immunoglobulin antibody labeled with a reporter molecule.

22. A method for detecting a MUC13 gene in a cell extract said method comprising contacting said extract with a MUC13 specific probe or primer for a time under conditions sufficient for the probe or primer to hybridize to said MUC13 gene and then detecting the presence of hybridization.

23. A method according to claim 22 wherein hybridization is detected by an amplification reaction.

24. A method of defining specific haematopoietic cell populations which employs an antibody, probe or primer as defined above to detect expression of MUC13. The MUC13 expressing cells can then be purified or eliminated from haematopoietic cell populations, including for the purpose of modifying bone marrow cell populations Prior to transplantion.

25. A method of detecting whether a patient has a predisposition to cancer or a related condition which comprises the step of detecting the presence or absence of an alteration in the gene encoding MUC13, wherein the presence of an alteration is indicative of a predisposition to cancer.

26. A genetically modified animal carrying a mutation in one or both alleles of an equivalent or homologue or relative of the human MUC13 gene.

27. A genetically modified animal according to claim 26 wherein the animal is a mouse.

28. A composition comprising a protein according to claims 1 to 6 and one or more pharmaceutically acceptable carriers and/or diluents.

29. A modulator of a protein according to any one of claims 1 to 6.

30. A modulator according to claim 29 wherein the modulator is an antagonist.

31. A modulator according to claim 29 wherein the modulator is an agonist.

32. A composition comprising a modulator according to claim 29 to 31 and one or more pharmaceutically acceptable carriers and/or diluents.

33. Use of a modulator of MUC13 in a manufacture of a medicament for the treatment of cancer or epithelial or haematopoietic malignant or non-malignant disease conditions.

34. Use of MUC13 in a manufacture of a medicament for the treatment, of cancer or epithelial or haematopoietic malignant or non-malignant disease conditions.
